(19) United States
(12) Patent Application Publication
(10) Pub. No.: US 2003/0199531 A1
(43) Pub. Date: Oct. 23, 2003

(54) YIGSR peptidomimetics and methods for using the same

(76) Inventors: Todd Wipke, Santa Cruz, CA (US); Jim Arnold, Berkeley, CA (US); John Lawton, Santa Cruz, CA (US); Jean Starkey, Bozeman, MT (US); Joseph Paul Korpelski, Santa Cruz, CA (US); Stephanie Hopkins, Poway, CA (US); Felix Busque Sanchez, Barcelona (ES)

Correspondence Address: BOZICEVIC, FIELD & FRANCIS LLP, 200 MIDDLEFIELD RD, SUITE 200, MENLO PARK, CA, 94025, US

(21) Appl. No.: 10/200,076

(22) Filed: Jul. 19, 2002

Related U.S. Application Data
(60) Provisional application No. 60/306,952, filed on Jul. 19, 2001.

(51) Int. Cl. A61K 31/519; C07D 487/14

(52) U.S. Cl. 514/267; 544/250

(57) ABSTRACT

YIGSR peptidomimetics and methods for making and using the same are provided. The subject YIGSR peptidomimetics are non-peptidic, biodegradation-resistant molecules that mimic the pentapeptide tyrosine-isoleucine-glycine-serine-arginine (YIGSR) binding site for the 67 kiloDalton (kDa) laminin binding protein (LBP). The subject peptidomimetics include a rigid or substantially rigid non-peptidic scaffold that effectively presents or positions a tyrosine or tyrosine-like group, an isoleucine or isoleucine-like group, a serine or serine-like group, and an arginine or arginine-like group in substantially the same manner as occurs in the YIGSR peptide itself. The subject peptidomimetics find use in a variety of different applications, including diagnostic and therapeutic applications.

### CROSS-REFERENCE To RELATED APPLICATIONS

Pursuant to 35 U.S.C. §119 (e), this application claims priority to the filing date of U.S. Provisional Application Serial No. 60/306,952 filed on Jul. 19, 2001; the disclosure of which is herein incorporated by reference.

### STATEMENT REGARDING FEDERALLY SPONSORED RESEARCH

This invention was made with government support under grant no. 2R01-EY06913-01 awarded by the National Institute of Health, grant no. DAMD-17-97-1-7207-B awarded by the U.S. Army, and grant no. #3CB-0183 awarded by the University of California. The government may have certain rights in this invention.

### INTRODUCTION

### BACKGROUND OF THE INVENTION

The metastasis of cells from primary tumors to distant sites within the body is a major cause of cancer mortality. The glycoprotein laminin is a component of the extracellular matrix that promotes cell adhesion, migration, proliferation, differentiation, phagocytosis, collagenase production, neurite outgrowth, and tumor cell invasiveness. Laminin contains multiple sites for interactions with other basement membrane components such as collagen IV, nidogen and heparin sulfate proteoglycan, as well as cell adhesion molecules such as integrin and non-integrin laminin receptors, including the 67 kDa laminin binding protein (LBP). Tumor cells with surface laminin receptors bind and attach to laminin more readily than normal cells. An active site for cell adhesion via binding to LBP has been identified on the laminin β-1 chain that includes the nonapeptide CDPGYIGSR, known as “peptide 11”, and its C-terminal pentapeptide peptide tyrosine-isoleucine-glycine-serine-arginine (YIGSR).

Clinical studies on solid tumors have shown a positive correlation of high expression of the 67 kDa LBP with poor prognosis and unfavorable clinical outcomes. The YIGSR peptide and various peptides including the YIGSR sequence have been proposed and investigated for potential antitumor and antimetastasis therapies. YIGSR-containing bioactive peptides have been shown to block angiogenesis, alter the formation of capillary structures by endothelial cells, prevent formation of excess blood vessels in tissue, and inhibit in vivo tumor cell colonization of tissues. Particularly, by interacting with laminin receptors on malignant cells, the YIGSR-containing peptides block binding to laminin and limit the invasiveness of malignant cells. Such bioactive peptides, however, have poor stability in vivo due to enzymatic degradation and rapid renal excretion from the blood. Thus, large amounts of YIGSR or YIGSR-containing peptide have been required to obtain an inhibitory effect in vivo. The poor stability of YIGSR-containing peptides has hindered the development of therapies based on these peptides.

Peptide analogs of YIGSR have been prepared wherein selected residues are substituted or derivatized in order to identify the minimal residues required for bioactivity. Such analogs include YIGSE, CDPGYIGSR amide, YIGSR amide, RSGIY amide, RGDSGYIGSR amide, and poly(YIGSR). With the exception of the YIGSR polymer, however, modified peptides have not provided in vitro activity comparable to or greater than YIGSR itself. Further, modified peptides have not provided improved stability to enzymatic degradation.

Another approach to enhance the potency of bioactive peptides has been via bioconjugation with polymers such as chitosan, polyethylene glycol (PEG), polyvinyl pyrrolidone (PVP) and polystyrene-maleic acid (SMA). Bioconjugation of YIGSR peptides with such polymers has generally prolonged the blood residency of the peptides. While the inhibitory effect of such bioconjugated peptides can be enhanced due to the longer blood residency imparted by increased stability to peptidases in the blood, the specific activity of such bioconjugated peptides is generally decreased, presumably due to steric hindrance by the attached polymer which inhibits receptor binding. Further, while the plasma half-life of bioconjugated peptides is increased with respect to YIGSR peptide itself, the bioconjugated peptides are still subject to relatively rapid biodegradation.

There is accordingly a need for a class of molecules which mimic the YIGSR ligand binding site for the laminin binding protein and which exhibit the antimetastasis properties of YIGSR peptide, but which are resistant to biodegradation and provide long blood residence for useful antitumor and antimetastasis therapies. The present invention satisfies this needs, as well as others, and generally overcomes the deficiencies found in the background art.

### SUMMARY OF THE INVENTION

YIGSR peptidomimetics and methods for making and using the same are provided. The subject YIGSR peptidomimetics are non-peptidic, biodegradation-resistant molecules that mimic the pentapeptide tyrosine-isoleucine-glycine-serine-arginine (YIGSR) binding site for the 67 kiloDalton (kDa) laminin binding protein (LBP). The subject peptidomimetics include a rigid or substantially rigid non-peptidic scaffold that effectively presents or positions a tyrosine or tyrosine-like group, an isoleucine or isoleucine-like group, a serine or serine-like group, and an arginine or arginine-like group in substantially the same manner as occurs in the YIGSR peptide itself. The subject peptidomimetics find use in a variety of different applications, including diagnostic and therapeutic applications.

### BRIEF DESCRIPTIONS OF THE DRAWINGS

FIG. 1A is a stereoview illustration of the s-conformation of the cysteine-bridged laminin peptide 11.

FIG. 1B is a stereoview illustration of a w-conformation of the cysteine-bridged laminin peptide 11.

FIG. 2A is a stereoview illustration the conformation of a peptidomimetic molecule in comparison with the s-conformation of the YIGSR peptide.

FIG. 2B is a stereoview illustration of the conformation of the peptidomimetic molecule of FIG. 2A in comparison with the w-conformation of the YIGSR peptide.

FIGS. 3A and FIG. 3B illustrate a synthetic route for a peptidomimetic molecule in accordance with the present invention.

### DEFINITIONS

Unless otherwise stated, the following terms used in the specification and claims have the meanings given below:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkenyl” means a linear monovalent hydrocarbon radical of two to six carbon atoms or a branched monovalent hydrocarbon radical of three to six carbon atoms containing at least one double bond, e.g., ethenyl, 2-propenyl, and the like.

“Alkynyl” means a linear monovalent hydrocarbon radical of two to six carbon atoms or a branched monovalent hydrocarbon radical of three to six carbon atoms containing at least one triple bond, e.g., ethynyl, propynyl, butynyl, and the like.

“Cycloalkyl” means a cyclic saturated monovalent hydrocarbon radical of three to seven carbon atoms, e.g., cyclopropyl, cyclohexyl, and the like.

“Halo” means fluoro, chloro, bromo, and iodo.

“Haloalkyl” means alkyl substituted with one or more halogen atoms, including those substituted with different halogens, e.g., —CH₂Cl, —CF₃, —CH₂CF₃, —CF₂CF₃, —CH₂CCl₃, and the like.

“Alkoxy”, “alkenyloxy”, “cycloalkyloxy”, or “haloalkyloxy” means a radical —OR where R is alkyl, alkenyl, cycloalkyl, or haloalkyl respectively as defined above, e.g., methoxy, ethoxy, propoxy, 2-propoxy, ethenyloxy, cyclopropyloxy, cyclobutyloxy, —OCH₂ Cl, —OCF₃, and the like.

“Alkylthio” or “cycloalkylthio” means a radical —SR where R is alkyl or cycloalkyl respectively as defined above, e.g., methylthio, butylthio, cyclopropylthio, and the like.

“Acyl” means a radical —C(O)R where R is hydrogen, alkyl, or haloalkyl as defined above, e.g., formyl, acetyl, trifluoroacetyl, butanoyl, and the like.

“Amino” means a radical —NH₂, (1-methylethyl)amino, and the like.

“Disubstituted amino” means a radical —NRR′ where R and R′ are independently alkyl or acyl, e.g.,dimethylamino, methylethylamino, di(1-methylethyl)amino, and the like.

“Hydroxyalkyl” means a linear monovalent hydrocarbon radical of two to six carbon atoms or a branched monovalent hydrocarbon radical of three to six carbons substituted with one or two hydroxy groups, provided that if two hydroxy groups are present they are not both on the same carbon atom. Representative examples include, but are not limited to, 2-hydroxyethyl, 2-hydroxypropyl, 3-hydroxypropyl, 1-(hydroxymethyl)-2-methylpropyl, 2-hydroxybutyl, 3-hydroxybutyl, 4hydroxybutyl, 2,3-dihydroxypropyl, 1-(hydroxymethyl)-2-hydroxyethyl, 2,3-dihydroxybutyl, 3,4dihydroxybutyl and 2-(hydroxymethyl)-3-hydroxypropyl, 2-hydroxyethyl, 2,3-dihydroxypropyl, and 1-(hydroxymethyl)-2-hydroxyethyl.

“Alkoxyalkyl” means a linear monovalent hydrocarbon radical of one to six carbon atoms or a branched monovalent hydrocarbon radical of three to six carbons substituted with at least one alkoxy group as defined above, e.g., 2-methoxyethyl, 2-methoxypropyl, and the like.

“Hydroxyalkyloxy” or “alkoxyalkyloxy” means a radical-OR where R is hydroxyalkyl or alkoxyalkyl respectively as defined above, e.g., 2-hydroxyethyloxy, 2-methoxyethyloxy, and the like.

“Aminoalkyl” means a linear monovalent hydrocarbon radical of two to six carbon atoms or a branched monovalent hydrocarbon radical of three to six carbons substituted with at least one —NRR′, where R and R′ are independently selected from hydrogen, alkyl, or acyl e.g., 2-aminoethyl, 2-N,N-diethylaminopropyl, 2-N-acetylaminoethyl, and the like.

“Aryl” means a monovalent monocyclic or bicyclic aromatic hydrocarbon radical of 6 to 12 ring atoms, and optionally substituted independently with one or more substituents selected from alkyl, haloalkyl, cycloalkyl, alkoxy, alkylthio, halo, nitro, acyl, cyano, amino, monosubstituted amino, disubstituted amino, -hydroxy, carboxy, or alkoxycarbonyl. Representative examples include, but are not limited to, phenyl, biphenyl, 1-naphthyl, and 2-naphthyl and the derivatives thereof.

“Heteroaryl” means a monovalent monocyclic or bicyclic aromatic radical of 5 to 10 ring atoms containing one or more, sometimes one or two ring heteroatoms selected from N, O, or S, the remaining ring atoms being C. The heteroaryl ring is optionally substituted independently with one or more substituents, sometimes one or two substituents, selected from alkyl, haloalkyl, cycloalkyl, alkoxy, alkylthio, halo, nitro, acyl, cyano, amino, monosubstituted amino, disubstituted amino, hydroxy, carboxy, or alkoxycarbonyl. Specifically the term heteroaryl includes, but is not limited to, pyridyl, pyrrolyl, thienyl, furanyl, indolyl, quinolyl, benzopyranyl, and thiazolyl, and the derivatives thereof. “Heterocycloamino” means a saturated monovalent cyclic group of 3 to 8 ring atoms, wherein at least one ring atom is N and optionally contains a second ring heteroatom selected from the group consisting of N, O, or S(O) ₙ (where n is an integer from 0 to 2), the remaining ring atoms being C. The heterocycloamino ring may be optionally fused to a benzene ring or it may be optionally substituted independently with one or more substituents, sometimes one or two substituents, selected from alkyl, haloalkyl, cycloalkyl, cycloalkylalkyl, aryl, aralkyl, heteroaryl, heteroaralkyl, halo, cyano, acyl, amino, monosubstituted amino, disubstituted amino, carboxy, or alkoxycarbonyl. More specifically the term heterocycloamino includes, but is not limited to, pyrrolidino, piperidino, morpholino, piperazino, indolino, and thiomorpholino, and the derivatives thereof.

“Heterocyclo” means a saturated monovalent cyclic group of 3 to 8 ring atoms in which one or two ring atoms are heteroatoms selected from N, O, or S(O)ₙ, where n is an integer from 0 to 2, the remaining ring atoms being C. The heterocyclo ring may be optionally fused to a benzene ring or it may be optionally substituted independently with one or more substituents, sometimes one or two substituents, selected from alkyl, haloalkyl, cycloalkyl, cycloalkylalkyl, aryl, aralkyl, heteroaralkyl, halo, cyano, acyl, monosubstituted amino, disubstituted amino, carboxy, or alkoxycarbonyl. More specifically the term heterocyclo includes, but is not limited to, pyrrolidino, piperidino, morpholino, piperazino, tetrahydropyranyl, and thiomorpholino, and the derivatives thereof.

“Cycloalkylalkyl” means a radical —R^{a} R^{b} where R^{a} is an alkylene group and R^{b} is a cycloalkyl group as defined above e.g., cyclopropylmethyl, cyclohexylpropyl, 3-cyclohexyl-2-methylpropyl, and the like.

“Cycloalkylalkyloxy” means a radical —OR where R is a cycloalkylalkyl group as defined above e.g., cyclopropylmethyloxy, 3-cyclohexylpropyloxy, and the like.

“Aralkyl” means a radical —R^{a} R^{b} where R^{a} is an alkylene group and R^{b} is an aryl group as defined above e.g., benzyl, phenylethyl, 3-(3-chlorophenyl)-2-methylpentyl, and the like.

“Heteroaralkyl” means a radical —R^{a} R^{b} where R^{a} is an alkylene group and R^{b} is a heteroaryl group as defined above e.g., 2-, 3-, or 4-pyridylmethyl, furan-2-ylmethyl and the like.

“Heterocycloalkyl” means a radical —R^{a} R^{b} where R^{a} is an alkylene group and R^{b} is a heterocyclo group as defined above e.g., morpholin-4-ylethyl, tetrahydrofuran-2-ylmethyl and the like.

“Pro-drugs” means any compound which releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited-to esters (e.g.; acetate, formate, and benzoate derivatives), carbamates (e.g., N,N-dimethylaminocarbonyl) of hydroxy functional groups in compounds of formula (I), and the like.

“Optional” or “optionally” means that the subsequently described event or circumstance may, but need not, occur, and that the description includes instances where the event or circumstance occurs and instances in which it does not. For example, “heterocyclo group optionally mono- or di- substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4methylbicyclo-2,2,2′oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

A:“therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “peptidomimetic” as used herein means, a non-peptide, molecule having an assembly of amino acid side chains or pharmacophores, or suitable derivatives thereof, that are supported on a substantially rigid, low molecular weight scaffold such that the spatial orientation of the pharmacophores substantially mimic the bioactive conformation of a parent or “mimicked” peptide.

The term “scaffold” as used herein means generally a rigid or substantially rigid molecular backbone, skeleton, or structure usable for positioning of amino acid or amino acid-like groups, and which is resistant to biodegradation under physiological conditions.

The terms “tyrosine-like group”, “tyrosine”, “tyrosine group” “tyrosine moiety”, “Tyr” or “tyr” mean tyrosine or a tyrosine residue, or any group which presents a phenol functional group or structure in a manner similar to that of tyrosine. Such tyrosine-like groups may comprise, for example, an alkyl phenol, alkanoyl phenol, amido phenol or like group which suitably positions a 4-hydroxyphenyl group with respect to the substantially rigid scaffold portion of a peptidomimetic molecule.

The terms “isoleucine-like group”, “isoleucine”, “isoleucine group”, “isoleucine moiety”, “Ile” or “ILe” mean isoleucine or an isoleucine residue, or any group which presents an alkyl functional group or structure similar to isoleucine. Such isoleucine-like groups include, for example, isobutyl, isopropyl, 3-methylpropyl, ethyl and methyl groups, as well as like short chain straight or branched alkyl group which presents an alkyl moiety similar to isoleucine with respect to the substantially rigid scaffold portion of a peptidomimetic molecule.

The terms “serine-like group”, “serine”, “serine group”, “serine moiety”, “Ser” or “ser” mean serine or a serine residue, or any group which presents a hydroxyalkyl or like functional group or structure in a manner similar to that of serine. Such serine-like groups may comprise, for example, a hydroxy, hydroxy methyl or other hydroxyalkyl group which presents a hydroxyl moiety in a manner similar to serine with respect to the substantially rigid scaffold portion of a peptidomimetic molecule.

The terms “arginine-like group”, “arginine, “arginine group”, “arginine moiety”, “arg” or “Arg” mean arginine or an arginine residue, or any functional group which presents an imine and/or amine, diamine, alkylamine, or like functional group or structure in a manner similar to that of arginine with respect to the substantially rigid scaffold portion of a peptidomimetic molecule.

### DESCRIPTION OF THE SPECIFIC EMBODIMENTS

YIGSR peptidomimetics and methods for making and using the same are provided. The subject YIGSR peptidomimetics are non-peptidic, biodegradation-resistant molecules that mimic the pentapeptide tyrosine-isoleucine-glycine-serine-arginine (YIGSR) binding site for the 67 kiloDalton (kDa) laminin binding protein (LBP). The subject peptidomimetics include a rigid or substantially rigid non-peptidic scaffold that effectively presents or positions a tyrosine or tyrosine-like group, an isoleucine or isoleucine-like group, a serine or serine-like group, and an arginine or arginine-like group in substantially the same manner as occurs in the YIGSR peptide itself. The subject peptidomimetics find use in a variety of different applications, including diagnostic and therapeutic applications.

Before the present molecules which mimic the YIGSR ligand are described, it should be understood that this invention is not limited to the particular molecular structures described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It should be noted that as used herein and in the appended claims, the singular forms “a”, “and”, and “the” include plural referents unless the context clearly dictates otherwise. Thus, for example, reference to “a molecule” or “compound” includes a plurality of such molecules or compounds, and reference to “the molecule” or “the compound” includes reference to one or more such molecules or compounds and equivalents thereof known to those skilled in the art, and so forth.

Any publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the subject peptidomimetic compounds are described first in greater detail, followed by a review of various methods of producing the subject compounds as well as representative applications in which the subject compounds find use.

YIGSR Peptidomimetic Compounds

The peptidomimetic molecules of the invention have, as a general structure, a rigid or substantially rigid non-peptidic scaffold that effectively presents or positions a tyrosine or tyrosine-like group, an isoleucine or isoleucine-like group, a serine or serine-like group, and an arginine or arginine-like group in substantially the same manner as occurs in the YIGSR peptide itself.

The rigid or substantially rigid scaffold portion may comprise a bicyclic, tricyclic or higher polycyclic carbon or heteroatom skeleton with a plurality of substitution sites suitably located for positioning of a tyrosine or tyrosine-like group, an isoleucine or isoleucine-like group, a serine or serine-like group, and an arginine or arginine-like group in a manner that simulates the arrangement of the tyrosine, isoleucine, serine and arginine residues in the YIGSR peptide. The substantially rigid scaffold portion of the molecule is one that is resistant to biodegradation under physiological conditions in many embodiments, as determined using any convenient biodegradation assay.

The peptidomimetic molecules of the invention may be shown generally by the structure

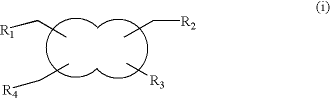

wherein a substantially rigid scaffold or skeleton portion of the molecule presents or positions R₁, R₂, R₃ and R4 groups, wherein R₁, R₂, R₃ and R4 respectively comprise arginine or an arginine-like group, tyrosine or a tyrosine-like group, isoleucine or an isoleucine-like group, and serine or a serine-like group.

More specifically, in the above structure:

The R₁ group of compound (i) may comprise arginine or an arginine residues, or any group which presents an imine and/or diamine structure in a manner similar to that of arginine.

The R₂ group may comprise tyrosine or a tyrosine residue, or any group which presents a phenol structure in a manner similar to that of tyrosine. Such tyrosine-like groups may comprise, for example, an alkyl phenol, alkanoyl phenol, amido phenol or like group which suitably positions a 4-hydroxyphenyl group with respect to the substantially rigid scaffold portion of the molecule.

The R₃ group may comprise isoleucine or an isoleucine residue, or any group which presents an alkyl structure similar to isoleucine. Isoleucine-like groups include, for example, isobutyl, isopropyl, 3-methylpropyl, ethyl and ethyl groups, as well as like short chain straight or branched alkyl group which presents an alkyl moiety similar to isoleucine with respect to the substantially rigid scaffold portion of the molecule.

The R4 group may comprise serine or a serine residue, or any group which presents an alkoxy structure in a manner similar to that of serine. Serine-like groups may comprise a hydroxy, hydroxy methyl or like group which presents a hydroxyalkyl moiety similar to serine with respect to the substantially rigid scaffold portion of the molecule, as well as other like groups.

The various peptidomimetic molecules of the invention generally fall within, or are subsets, i.e., species, of, the structure (i) above.

In certain embodiments, the peptidomimetic molecules of the invention may include a tri-cyclic scaffold and be described by the following structure:

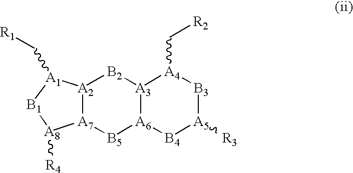

wherein A₁-A₈ may comprise carbon or nitrogen, B₁-B₅ may comprise carbon, nitrogen, oxygen or sulfur, and wherein R₁, R₂, R₃ and R4 respectively comprise arginine or an arginine-like group, tyrosine or a tyrosine-like group, isoleucine or an isoleucine-like group, and serine or a serine-like group. The compound or class of compounds represented by (ii) provide one specific subset of peptidomimetic molecules in accordance with the invention. Several specific peptidomimetic molecules fall within, or are subsets of, the structure (ii), as described further below.

In a more specific subset of the structure (iii) above, the peptidomimetic molecule of the invention may comprise the structure

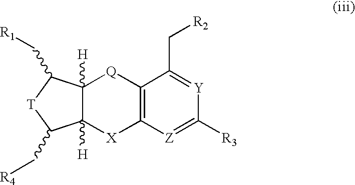

wherein Y and Z may comprise carbon or nitrogen, T, Q and X may comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

Still more specifically, several peptidomimetic compounds in accordance with the present invention are in structures (iv) through (xxv) below.

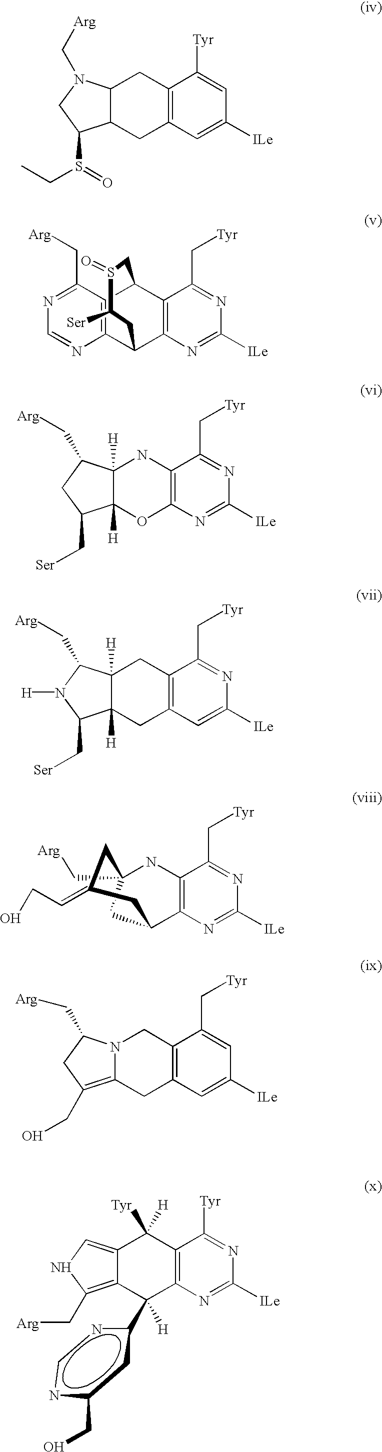

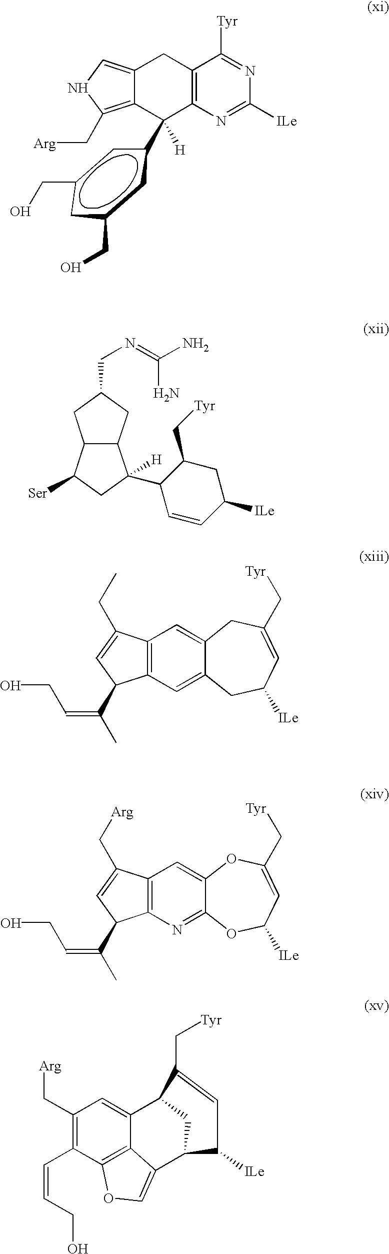

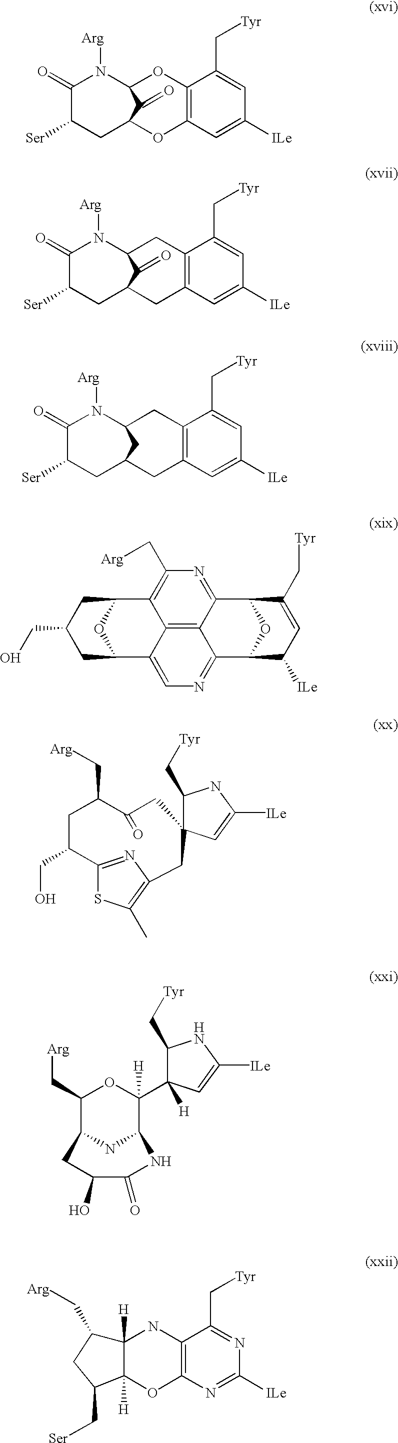

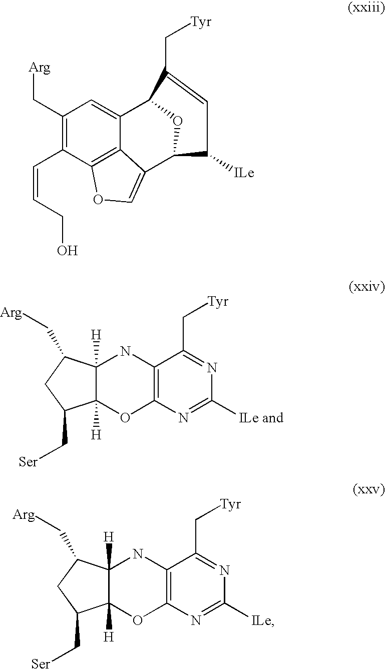

wherein the Arg-, Tyr-, Ile- and Ser-respectively represent arginine or an arginine-like group, tyrosine or a tyrosine-like group, isoleucine or an isoleucine-like group, and serine or a serine-like group. Of the above, compounds (iv), (vi), (vii), (ix), (xxii), (xxiii), (xxiv) and (xxv) are of particular interest in certain embodiments. These molecules are “subset” molecules of the structure (i), and more specifically of structure (ii), and still more specifically of structure (iii) above. The peptidomimetic molecule structure (iii) has a scaffold or skeleton comprising a generally linear tricyclic structure with an aromatic six-membered ring and an aliphatic five membered ring fused on opposite sides of an aliphatic six membered ring. Synthesis of peptidomimetic molecules of this type is described in the example provided below.

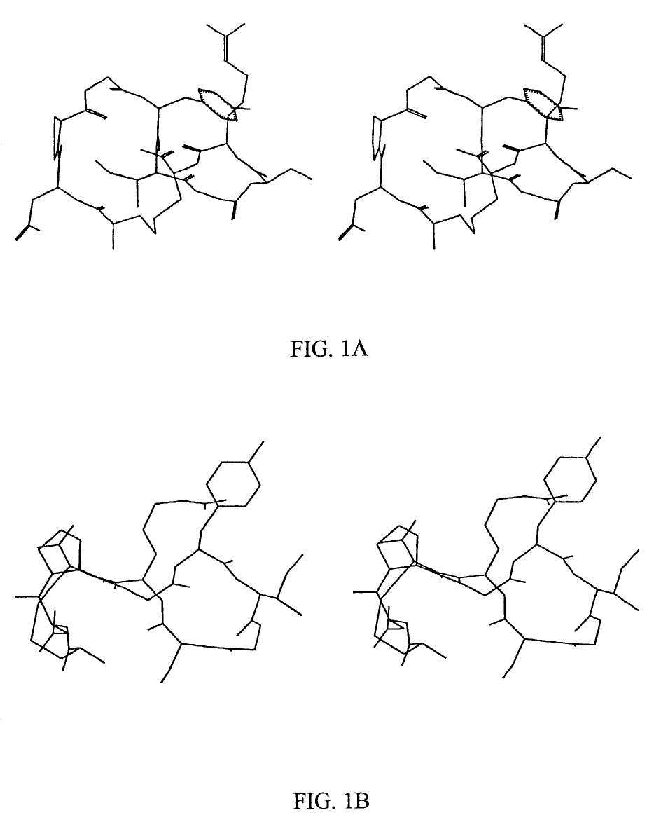

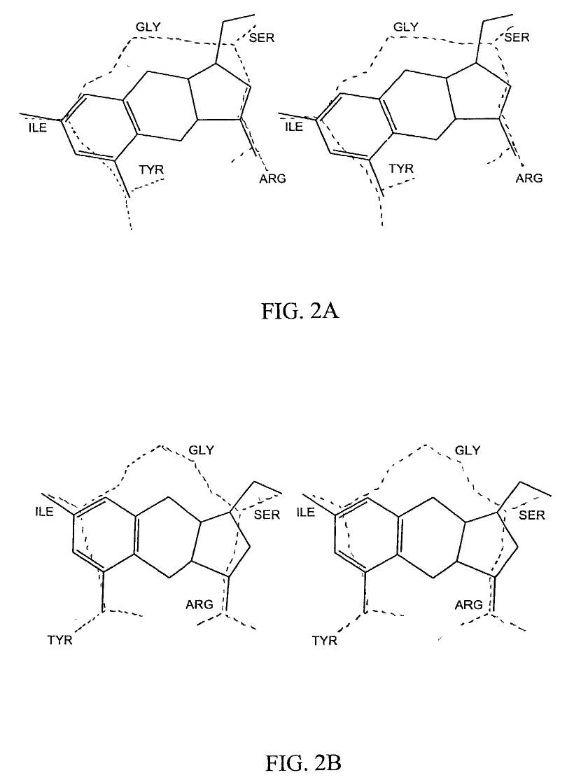

The “s-conformation” and “w-conformation” are the major conformations for the laminin peptide 11 (Ostheimer et al, J. Biol. Chem 1992 25120). The “w-conformation” shown in FIG. 1B is characterized by the “w” shape in the peptide backbone spanning the cysteine (i)-isoleucine (vi) residues. FIG. 2A and FIG. 2B illustrate respectively the superimposition of a non-functionalized scaffold corresponding to molecular structure (ii) (solid lines) with the YIGSR portion of the s-conformation and w-conformation of laminin peptide 11 (dashed lines). As can be seen, the scaffold arrangement of molecule (ii) provides positioning or placement of Arg-, Ser-, Tyr- and ILe-groups in substantially the same manner as occurs in the YIGSR peptide itself. The spacer or scaffold structure substantially matches both the C_{α}-C_{β} bond vectors of isoleucine and serine, and the main chain C_{α} bond for tyrosine and arginine. As noted above, compound (iii), as well as the specific peptidomimetic compounds (iv), (vi), (vii), (ix), (xxii), (xxiii), (xxiv) and (xxv) are subset molecules of the structure (ii). Superpositioning of the molecular structure (vi) (one particular version of the structure (iii) onto the s-conformation and w-conformations in the manner shown in FIG. 2A and FIG. 2B gives, for example, a root means squared deviation (RMSD) of 0.46 Angstroms and 0.49 Angstroms respectively for the s-conformation and w-conformations.

The substantially rigid scaffold of the subject peptidomimetic compounds allows accurate positioning of the arginine or arginine-like group, tyrosine or tyrosine-like group, isoleucine or isoleucine-like group, and serine or serine-like group, and provides for maintaining these groups in a conformation that provides high activity with regard to binding to the 67 kDa Laminin Binding Protein. Thus, certain embodiments of the invention provide in vitro activity comparable to or greater than the YIGSR peptide itself. Specifically, certain embodiments provide binding activity within the range of approximately that of the YIGSR peptide, to approximately 100 times the activity of YIGSR. Further, the non-peptiditic nature of the subject compounds makes the compounds resistant to biodegradation under physiological conditions, and facilitates maintenance of blood serum levels of the compounds when administered as pharmaceutical preparations in the manner described below.

With respect to the above representative structures, various other serine-like groups, tyrosine-like groups, isoleucine-like groups, and arginine-like groups will suggest themselves to those skilled in the art in view of the present disclosure, which alternatives fall within the scope of the present invention.

Likewise, with respect to the above representative structures, various additional scaffold structures suitable for positioning of Arg-, Tyr-, Ser- and ILe- groups on the peptidomimetic molecules of the invention will suggest themselves to those skilled in the art in view of this disclosure, and such additional scaffold structures are considered to be within the scope of this disclosure.

The non-peptidic structure of the peptidomimetic molecules of the invention present the same three-dimensional shape and orientation of side chains of the YIGSR pentapeptides, and present similar electrostatic fields to that of YIGSR, yet they do not rapidly metabolize like YIGSR and other bioactive peptides. The molecules of the invention mimic YIGSR in that they are configured for specific blocking of the binding of laminin and are relatively non-toxic.

The molecules of the invention can be modified to optimize binding and probe the electrostatic and steric requirements of the laminin binding site. The molecules of the invention may also be modified to allow attachment of chemical labels or probes for use in imaging or for targeted drug delivery agents, and such modified peptidomimetic compounds are considered part of the present invention.

General Synthetic Methods

The subject peptidomimetic compounds of the inventions may be synthesized using any convenient protocol or synthetic techniques. Several synthetic techniques usable for construction of polycyclic and heterocycle scaffold structures are known in the art and may be employed in addition to the ring fusion or coupling route used in the example provided below. The derivation or modification of the scaffold to provide a suitably positioned arginine-like group, serine-like group, tyrosine-like group and isoleucine-like group in accordance with the invention may utilize a variety of peptide and amino acid synthetic techniques known to those skilled in the art, including the formation of reactive hydroxyl, amino and/or methyl groups which are suitably protected during synthesis of the scaffold portion of the molecule, and which are subsequently deprotected and functionalized. A representative synthetic approach for compounds (ii) and (iii), and subset compounds thereof, is provided in the Experimental Section, infra.

Utility

The subject peptidomimetic compounds find utility in a variety of different applications, and in any application where YIGSR peptides find use. Representative applications include, but are not limited to: therapeutic applications, diagnostic applications, in the preparation of scientific research materials, and the like. Each of these representative applications is now described in greater detail.

Therapeutic Applications

The subject compounds find use in a variety of different therapeutic applications, where such applications are applications where administration of the subject compounds provides for a therapeutic outcome that may be based on a number of different mechanisms, including, but not limited to: blocking and thereby prevention of laminin binding to the laminin binding protein on certain target (such as cancer cells); selected or targeting delivery of various therapeutic agents (e.g., toxins) to certain target that display laminin binding protein on their surface (such as cancer cells), where the targeted agents are coupled, e.g., conjugated, to the subject peptidomimetic compounds; etc. In using the subject compounds in these types of applications, an effective amount of a compound, e.g., present in pharmaceutical preparation as described below, is administered to a host or subject in need thereof to achieve a desired result of the application, e.g., therapeutic result, such as treatment of the condition.

One therapeutic application in which the subject compounds find use is in the treatment of subjects having metastatic or hyperproliferative disorders, e.g. to inhibit tumor growth, to inhibit angiogenesis, to decrease inflammation associated with a lymphoproliferative disorder, to inhibit graft rejection, or neurological damage due to tissue repair, etc.

There are many disorders associated with a dysregulation of cellular proliferation, including hyperproliferation of blood vessels and epithelial hyperproliferative conditions associated with AIDS. Disorders which are treatable or potentially treatable with the subject petptidomimetic compounds include, by way of example, diabetic retinopathy, arthritis, particularly rheumatoid arthritis, psoriasis Kaposi sarcoma, newborn intra-vitreal neo-vascularization, pulmonary fibrosis, glaucoma, retinitis pigmentosa, and some forms of obesity. The conditions of interest also include, but are not limited to, the following conditions.

The subject methods apply to diseases where there is hyperproliferation and tissue remodelling or repair of reproductive tissue, e.g. uterine, testicular and ovarian carcinomas, endometriosis, squamous and glandular epithelial carcinomas of the cervix, etc. are reduced in cell number by administration of the subject compounds.

Tumor cells are characterized by uncontrolled growth, invasion to surrounding tissues, and metastatic spread to distant sites. Growth and expansion requires an ability not only to proliferate, but also to down-modulate cell death (apoptosis) and activate angiogenesis to produce a tumor neovasculature. Angiogenesis may be inhibited by affecting the cellular ability to interact with the extracellular environment and to migrate, which is an integrin and laminin binding protein-specific function, or by regulating apoptosis of the endothelial cells. Integrins function in cell-to-cell and cell-to-extracellular matrix (ECM) adhesive interactions and transduce signals from the ECM to the cell interior and vice versa. Since these properties implicate integrin involvement in cell migration, invasion, intra- and extra-vasation, and platelet interaction, a role for integrins in tumor growth and metastasis is obvious.

Tumors of interest for treatment include carcinomas, e.g. colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue remodelling and repair. For example, the chronic skin inflammation of psoriasis is associated with hyperplastic epidermal keratinocytes as well as infiltrating mononuclear cells, including CD4+memory T cells, neutrophils and macrophages.

The proliferation of immune cells is associated with a number of autoimmune and lymphoproliferative disorders. Diseases of interest include multiple sclerosis, rheumatoid arthritis and insulin dependent diabetes mellitus. Evidence suggests that abnormalities in apoptosis play a part in the pathogenesis of systemic lupus erythematosus (SLE). Other lymphoproliferative conditions the inherited disorder of lymphocyte apoptosis, which is an autoimmune lymphoproliferative syndrome, as well as a number of leukemias and lymphomas. Symptoms of allergies to environmental and food agents, as well as inflammatory bowel disease, may also be alleviated by the compounds of the invention.

The subject methods are also applied to the treatment of a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

The present compounds are useful for therapeutic purposes, including prophylactic purposes. As used herein, the term “treating” is used to refer to both prevention of disease, and treatment of pre-existing conditions. The prevention of proliferation is accomplished by administration of the subject compounds prior to development of overt disease; e.g. to prevent the regrowth of tumors, prevent metastatic growth, diminish restenosis associated with cardiovascular surgery, etc. Alternatively the compounds are used to treat ongoing disease, by stabilizing or improving the clinical symptoms of the patient.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

Representative therapeutic applications in which the subject compounds find use are further described in U.S. Pat. Nos. 5,039,662; 5,211,657, 5,231,082 and 5,629,412; the disclosures of which are herein incorporated by reference.

Diagnostic Applications

The subject peptidomimetic compounds also find use in various diagnostic applications, in which the subject compounds are employed to detect the presence of cells that display laminin binding proteins on their surface in medium, e.g., in an in vitro sample or in an animal, e.g., subject or host. In such applications, the compounds, which may or may not be labeled with a suitable label, e.g., an isotopic label, a fluorescent label, etc., are contacted with the medium under conditions sufficient for the compounds to bind to the cells of interest, if present in the medium. Where the medium is an animal, the compounds are typically administered to the animal. The presence of any compounds bound to cells in the medium is then determined. A specific example of diagnostic applications is the detection/imaging of cancer cells in a subject. Representative diagnostic applications are further described in U.S. Pat. Nos. 5,556,609; 5,567,408; 5,681,541; 5,788,960; and 5,811,394; the disclosures of which are herein incorporated by reference.

Preparation of Compositions of Matter

The subject compounds also find use in the preparation of compositions of matter in which it is desired to have a YIGSR mimetic present. For example, the subject compounds find use in the preparation of research materials, e.g., plates, slides, etc., in which adhesion of cells to the surface thereof is desired, where the compounds are coated on the surface of such structures to provide for cellular adhesion. Further examples of such applications are provided in U.S. Pat. Nos. 5,211,567 and 5,643,561; the disclosures of which are herein incorporated by reference.

Pharmaceutical Preparations

Also provided are pharmaceutical preparations of the subject peptidomimetic compounds. The subject compounds can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. The formulations may be designed for administration via a number of different routes, including oral, buccal, rectal, parenteral, intraperitoneal, intradermal, transdermal, intracheal, etc., administration.

In pharmaceutical dosage forms, the peptidomimetic compounds may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the peptidomimetic compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The peptidomimetic compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The peptidomimetic compounds can be utilized in aerosol formulation to be administered via inhalation. The compounds of the present invention can be formulated into pressurized acceptable propellants such as dichlorodifluoromethane, propane, nitrogen and the like.

Furthermore, the peptidomimetic compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

Depending on the patient and condition being treated and on the administration route, the subject peptidomimetic compounds may be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

For use in the subject methods, the subject peptidomimetic compounds may be formulated with other pharmaceutically active agents, particularly other anti-metastatic, anti-tumor or anti-angiogenic agents. Angiostatic compounds of interest include angiostatin, endostatin, carboxy terminal peptides of collagen alpha (XV), etc. Cytotoxic and cytostatic agents of interest include adriamycin, alkeran, Ara-C, BICNU, busulfan, CNNU, cisplatinum, cytoxan, daunorubicin, DTIC, 5-FU, hydrea, ifosfamide, methotrexate, mithramycin, mitomycin, mitoxantrone, nitrogen mustard, velban, vincristine, vinblastine, VP- 16, carboplatinum, fludarabine, gemcitabine, idarubicin, irinotecan, leustatin, navelbine, taxol, taxotere, topotecan, etc.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular peptidomimetic compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific peptidomimetic compound, the severity of the symptoms and the susceptibility of the subject to side effects. Preferred dosages for a given peptidomimetic compound are readily determinable by those of skill in the art by a variety of means.

Kits with unit doses of the peptidomimetic compounds, usually in oral or injectable doses, are provided. In such kits, in addition to the containers containing the unit doses will be an informational package insert describing the use and attendant benefits of the drugs in treating pathological condition of interest. Preferred peptidomimetic compounds and unit doses are those described herein above.

The following examples are offered by way of illustration and not by way of limitation. The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Experimental

I. Synthesis of YIGSR Peptidomimetic Compounds

Compound (ii) above is composed of a rigid core that presents the sidechains of the key amino acids in the correct spatial orientation for maximum overlap with the defined conformation of the YIGSR portion of the laminin peptide 11 bound to LBP. Molecule (xxvi) below is a subset compound of (ii).

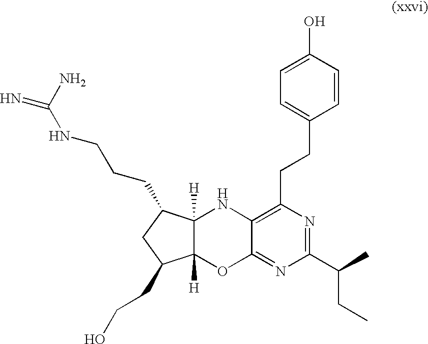

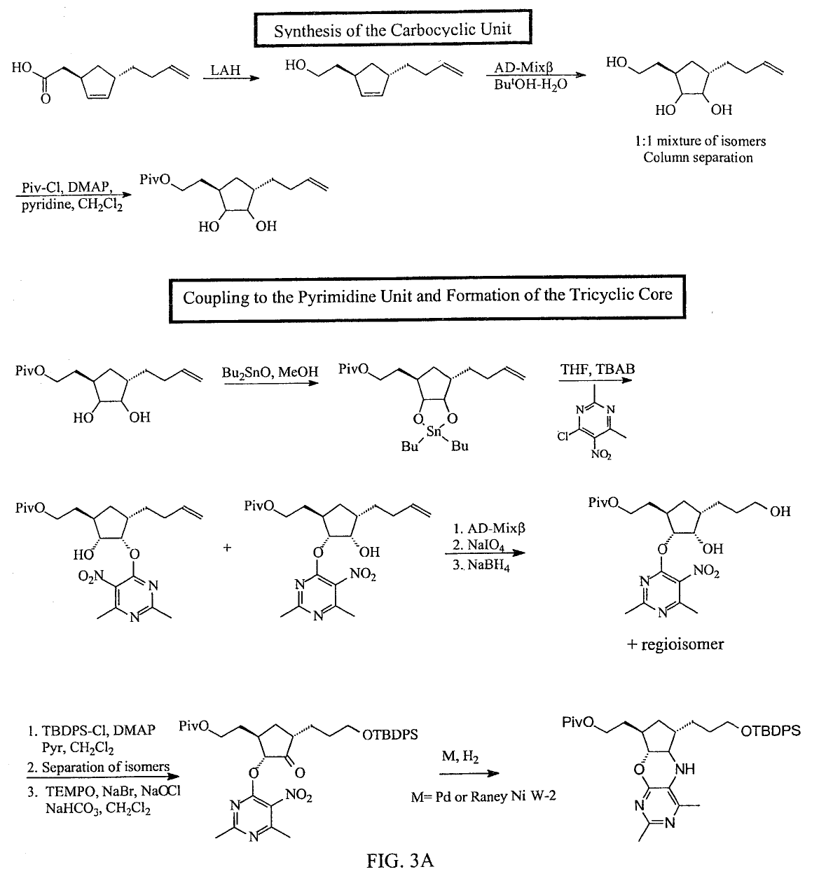

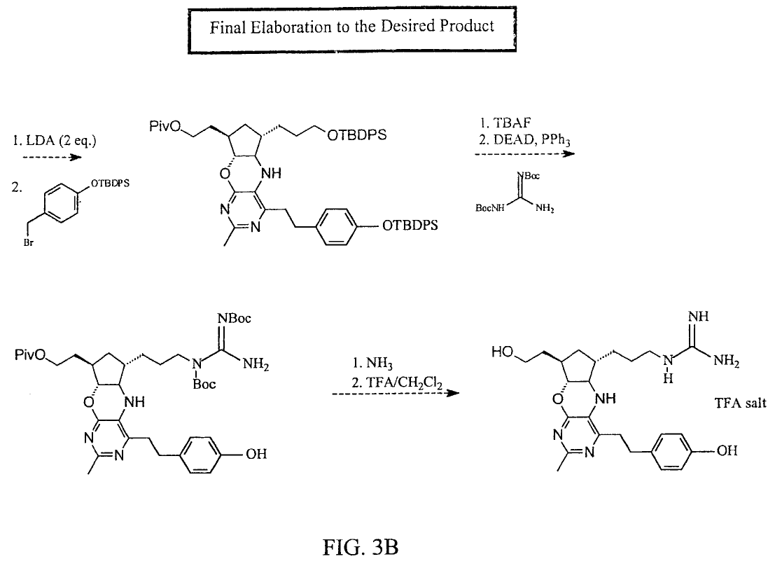

FIG. 3A and FIG. 3B illustrate generally the synthetic route for the peptidomimetic molecule (xxvi) in accordance with the invention.

The synthesis of structure (xxiv) in this example may be envisioned as coming from fragments 2 and 3 as shown below in the retrosynthesis of Scheme 1.

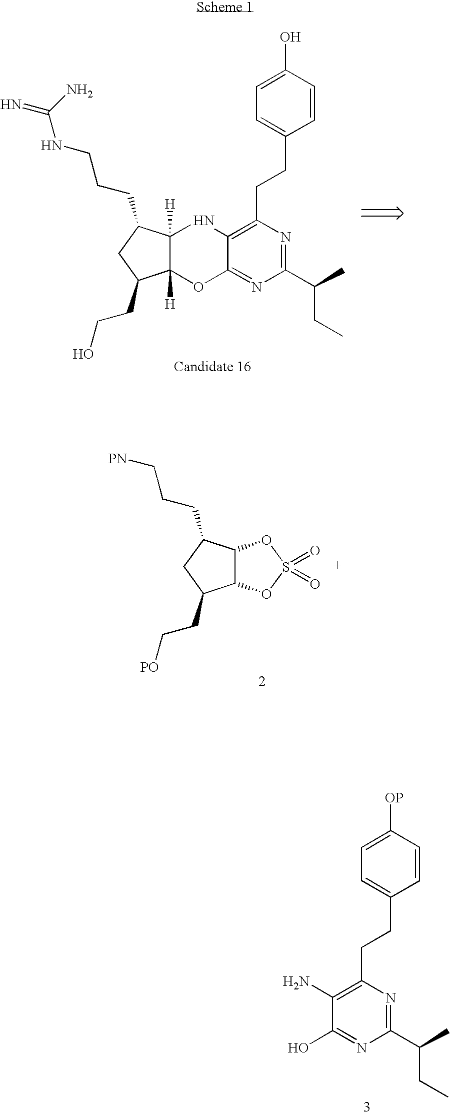

This convergent approach readily allows for the synthesis of molecule (vi) as well as derivative peptidomimetic molecules. For example, analogs of the pyrimidine moiety 3 could be readily generated, through primary synthesis of the heterocycle, using differently substituted starting materials. It was expected that, the primary alcohol or amine functionalities on carbocycle 2 could be easily substituted by or converted into appropriate mimics of the arginine or serine sidechains. Thus, coupling of 2 and 3, followed by deprotection of the protecting groups and elaboration to the guanidinylated compound, should produce the desired target peptidomimetic molecule (xiv) shown in Scheme 1.

The synthesis of a precursor of carbocycle 2, which contains four stereogenic centers, has been completed in a diastereoselective fashion starting from commercial 5-norbornen-2-ol 4 through the synthetic sequence outlined in Scheme 2.

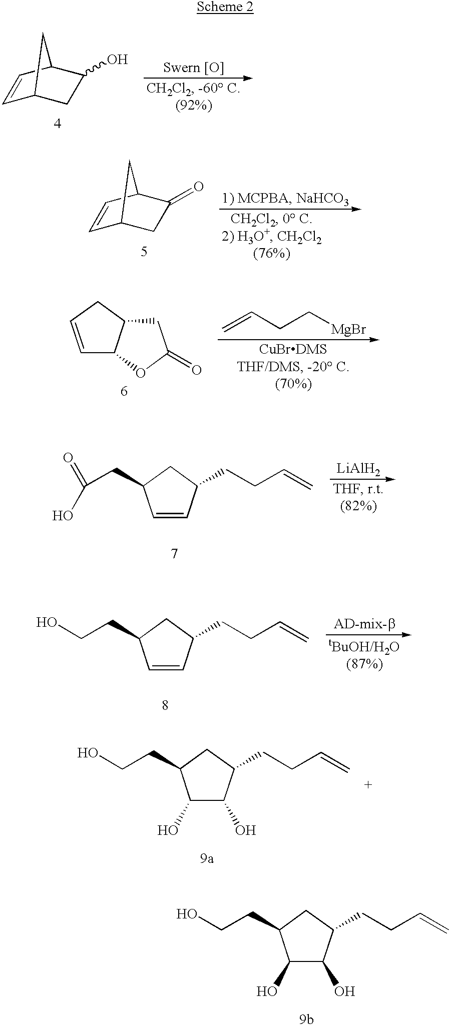

The synthesis shown in Scheme 2 starts with a nearly quantitative Swern oxidation of the alcohol 4 to ketone 5, followed by a Baeyer-Villager oxidation, which provides lactone 6 after rearrangement in acidic media, as shown in Scheme 3.

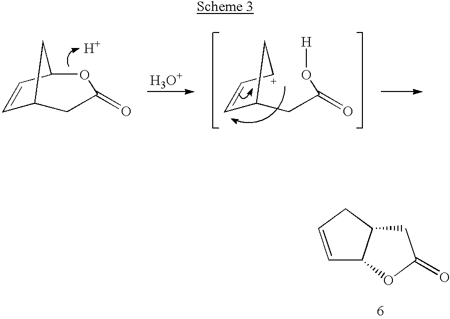

According to the procedure by Curran et al. in J. Org. Chem. 1986, 51, 1612-1613, unsaturated lactone 6 is regio- and stereoselectively opened by 3-butenyl magnesium bromide and copper(II)bromide, yielding the cyclopentene acid 7. The homolog, allyl magnesium bromide, proved unsuccessful in opening 6. As reported by Curran, the S_{N} 2′-anti selectivity is only achieved when a stoichiometric amount of CuBr-Me₂S is employed. Reduction of the acid functionality (vii) with lithium aluminum hydride provides alcohol 8.

Initially, a regioselective dihydroxylation of the terminal olefin in diene 8 was considered. The reaction was attempted using the commercially available osmium tetraoxide dihydroxylation reagent AD-mix (Kolb, H. C.; VanNieuwenhze, M. S.; Sharpless, K. B. Chem. Rev. 1994, 94, 2483-2547) since this reagent is generally selective for the least substituted double bond. Surprisingly, the internal olefin was selectively oxidized, resulting in a mixture of the corresponding diastereoisomeric triols 9a and 9b in a 1:1 ratio. Unexpectedly (Wipf, P.; Kim, Y.; Goldstein, D. M. J. Am. Chem. Soc. 1995, 117, 11106-11112; Andrus, M. B.; Lepore, S. D.; Turner, T. M. J. Am. Chem. Soc. 1997, J119, 12159-12169), no dihydroxylation of the terminal olefin was observed.

A fairly difficult separation of the isomers was accomplished by chromatography. Protecting the primary alcohol with various protecting groups did not result in an easier separation of the isomers. Compound 9a (26% from 8) was considered to be the desired isomer after careful analysis of ¹H NMR shifts. Diastereomer 9b was utilized as a model for 9a in subsequent steps. The primary hydroxyl of 9a or 9b was selectively converted to the corresponding tert-butyldiphenylsilyl ether by standard protocol (TPDPSCl, imidazole) (Overman, L. E.; Rishton, G. M. Org. Synth. 1992, 71, 56-61) as shown in Scheme 4 to yield the corresponding diol 10a or 10b (10 b not shown). Compound 10 a was used in preliminary coupling studies (vida infra).

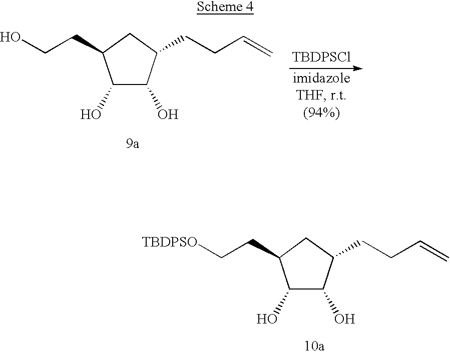

Because of the 5-amino group, the pyrimidine 3 proved to be very difficult to generate by primary synthesis. Generally, 5-amino pyrimidines are derived from the reduction of the corresponding 5-nitro compound. There is very little literature regarding other methods of incorporating the 5-amino functionality. Three possible synthetic routes were considered for the synthesis of this heterocycle (Brown, D. J. The Chemistry of Heterocyclic Compounds; the Pyrimidines, v. 52; John Wiley and Sons, Inc.; New York, 1994): (a) primary synthesis from an amidine and a alpha-nitrogen substituted β-keto ester, (b) primary synthesis of a 5-functionalized pyrimidine from which 3 could be elaborated, or (c) elaboration of a commercial pyrimidine.

A very standard method for generating pyrimidines is by a condensation between a substituted malonate or β-keto ester and an amidine. To our knowledge, there are no examples for generating a pyrimidine, in good yield, from a β-keto ester alpha substituted with a nitrogen containing group. As demonstrated in Scheme 5, condensation of the nitro, the bis-silyl amino or the phthalimide substituted β-keto ester 11 with isobutyl amidine 12 were all unsuccessful. Variation of the base and the solvent did not facilitate coupling.

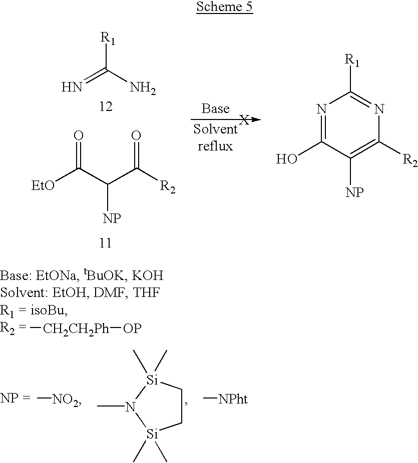

It was hypothesized that precursor 3 shown in Scheme 2 above could be generated from carboxylic acid intermediate 15 by a modified Curtius rearrangement (Ninomiya, K.; Shioiri, T.; Yamada, S. Tetrahedron 1974, 30.2151-2157. (b) Kim, D.; Weinreb, S. M. J. Org. Chem. 1978, 43, 125-131). Since primary synthesis of 15 or its ester homolog was not possible, compound 14 was generated from 12 and commercially available alpha-substituted allyl β-keto ester 13. Unfortunately all efforts to elaborate 14 were ineffective (Scheme 6).

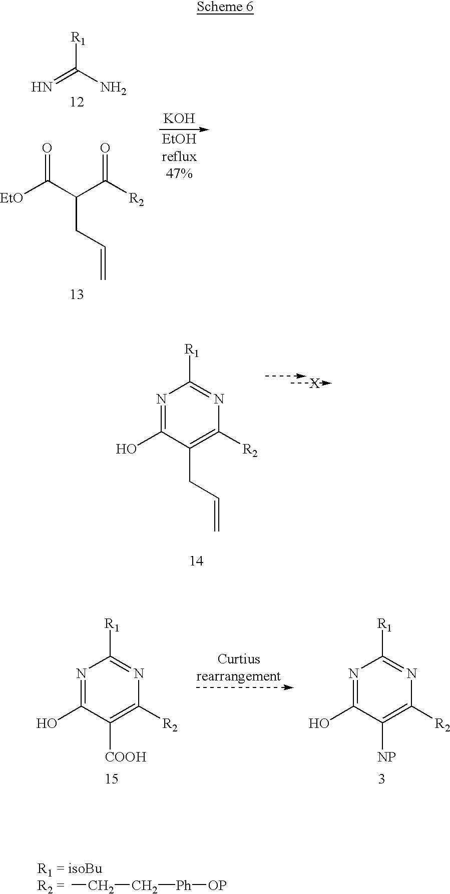

Ultimately, a less direct route to a precursor of 3 was taken. The simple pyrimidine, 5-amino-2,6-dimethyl-4-hydroxy pyrimidine 20 was readily available in a few steps by the literature procedure shown in Scheme 7 (Rose, F. L. J. Chem. Soc. 1954, 4116-4126; Albert, A.; Brown, D. J.; Wood, H. C. S. J. Chem. Soc. 1954, 3832-3839). Nitration of 4,6-dihydroxy-2-methyl-pyrimidine 16 followed by chlorination using phosphorus oxychloride affords the corresponding dichloronitropyrimidine 17. This heterocycle reacts with diethyl malonate in light petroleum in the presence of strong aqueous sodium hydroxide to furnish a red salt 18. Decomposition of 18 in hot hydrochloric acid yields the 2,6-dimethyl-4-hydroxy-5-nitropyrimidine 19. Reduction of the nitro group provides the corresponding aminopyrimidine 20.

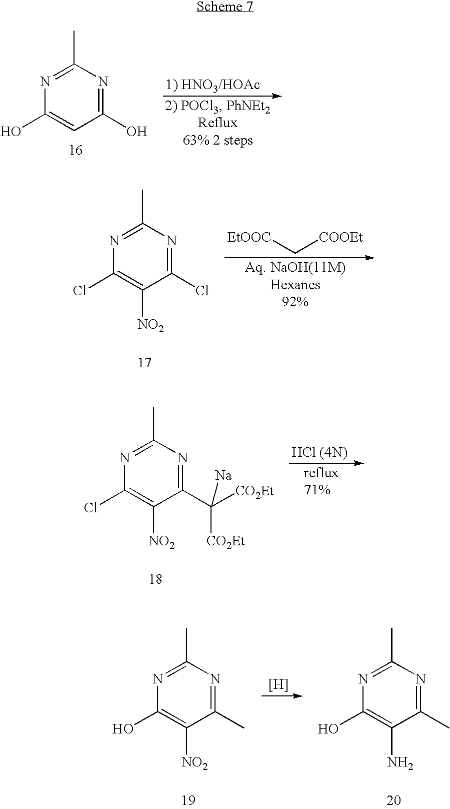

In an attempt to generate the tyrosinyl sidechain mimic, compound 18 was treated with benzyl bromide, as a model, in THF under reflux to yield 21 (Scheme 8). Compound 21 was also generated in 65% yield by a one-pot, two-step procedure where 17 was treated with diethyl malonate and NaH (2eq.) in THF followed by quenching with BnBr. Unfortunately, derivative 21 eluded decarboxylation. It should be noted that the dimethyl malonate derivative of both 18 and 21 also precludes decarboxylation. As a result, it was hoped that the dimethyl pyrimidine from 19 or 20 could be manipulated later in the synthesis to afford the appropriately substituted heterocycle.

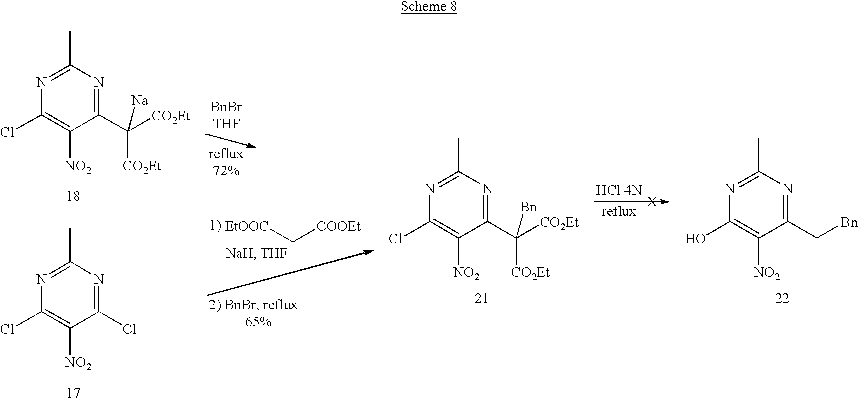

The validity of the coupling (Scheme 1) via nucleophilic displacement (S_{N}2) of the cyclic sulfate (fragment 2) by a 5-amino pyrimidine was tested using a commercial pyrimidine 23 and the sulfate 24 derived from 10a [SO₂(Im)₂] as shown in Scheme 9 (Berridge, M. S.; Franceschini, M. P.; Rosenfeld, E.; Tewson, T. J. J. Org. Chem. 1990, 55, 1211-1217). Surprisingly, the reaction did not work in THF or CH₃CN at reflux, or in DMF at 60 ° C.

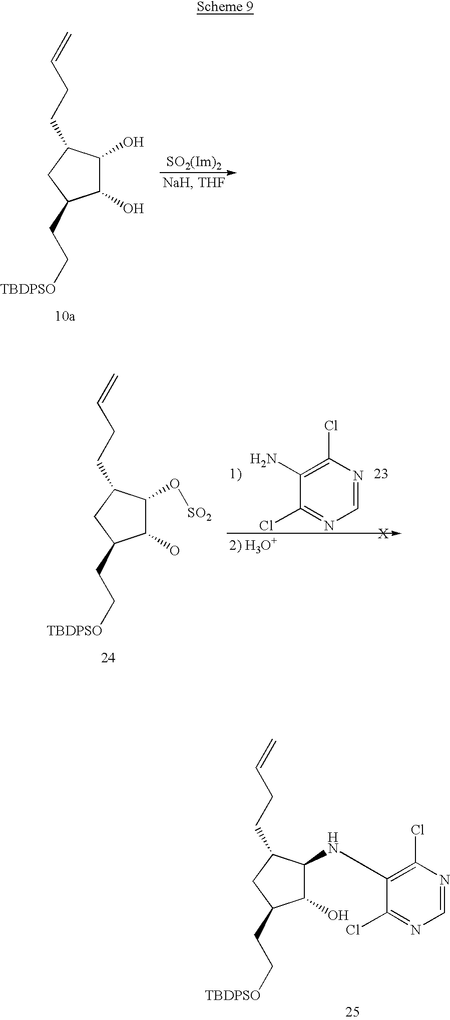

Alternatively, the monoalkoxide derived from diol 10a reacted with the 5-nitro pyrimidine 26 in an S_{N}Ar fashion to generate 27 in 17% yield (Scheme 10).

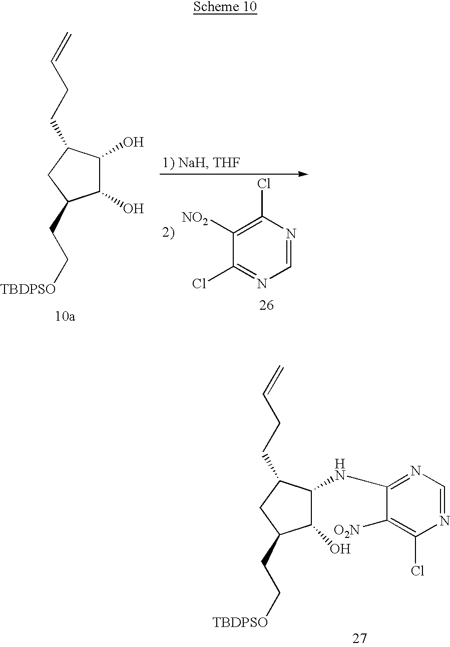

This result caused us to change our approach and utilize the umpulong (March, J. Advanced Organic Chemistry: Reactions, Mechanisms, and Structure, 4th ed.; John Wiley & Sons, 1992; p 471) conditions to provide the coupled material. After much experimentation, it was found that the cyclic stannylene derivative 28a coupled in good yield (65-85%) to pyrimidine 29 (made from POCl₃ treatment of 19) affording a mixture of regioisomers 30a and 30b (Kaji, E.; Harita, N. Tetrahedron Lett. 2000, 41, 53-56). Likewise, diastereomer 28b coupled to 29 to afford regioisomers 31a and 31b as shown in Scheme 11.

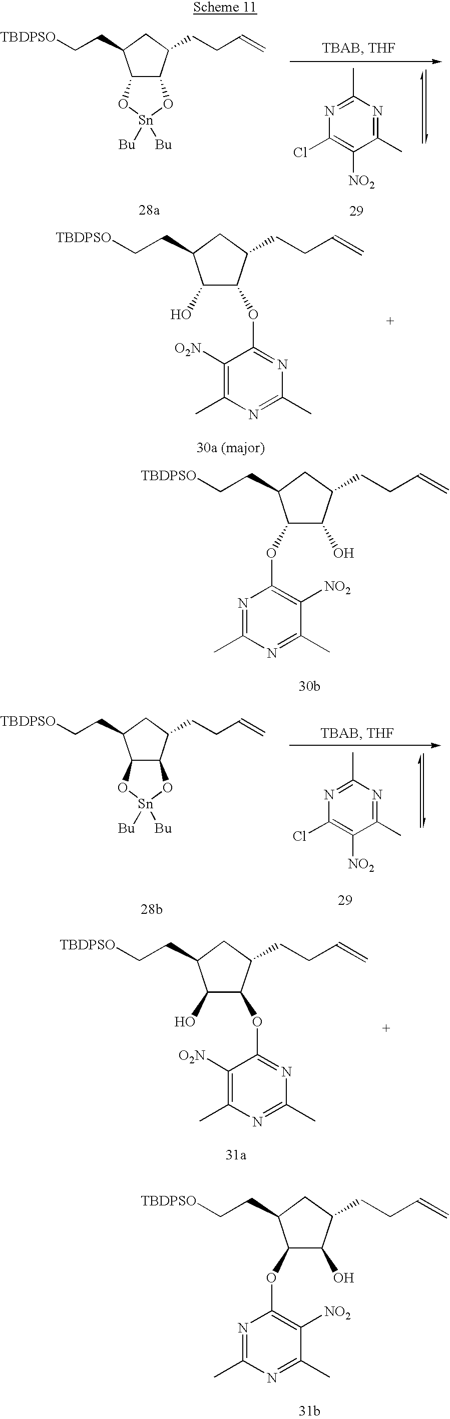

Although each regioisomer could be isolated by chromatography, two compounds were evident in the ¹H NMR. It was hypothesized that the regioisomers were interconverting as a result of the secondary alcohol acting as an intramolecular nucleophile on the nitro pyrimidine ring in a Smiles rearrangement (Okafor, C. O. J. Org. Chem. 1967,,38, 4386; Bunnett, J. F.; Zahler, R. E.; Chem. Rev. 1951, 49, 362; Truce, W. E. Org. Reactions 1970, 18, 99) in the manner shown in Scheme 12.

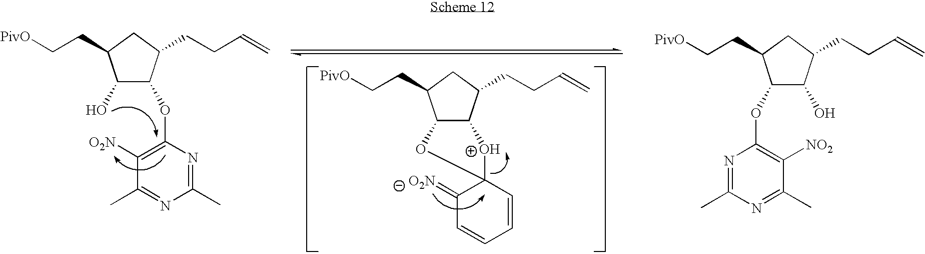

Once the coupled material (30a or 30b) was obtained in the above manner, attention was focused on generating the trans-fused 6-membered morpholino ring system of 3. It was hypothesized that the ring could be closed by an intramolecular S_{N}2 reaction between the 5-amino pyrimidine and the activated alcohol of 30a or 30b. Thus, the regioisomeric mixture 30a and 30b was treated with mesyl chloride and pyridine as shown in Scheme 13. Interestingly, only a single isomer 32 was obtained in an 87% crude yield (the pyridine hydrochloride salt was removed by trituration). Diastereomers 31a and 31b were subjected to the same conditions and again gave a single isomer 33 (Scheme 13).

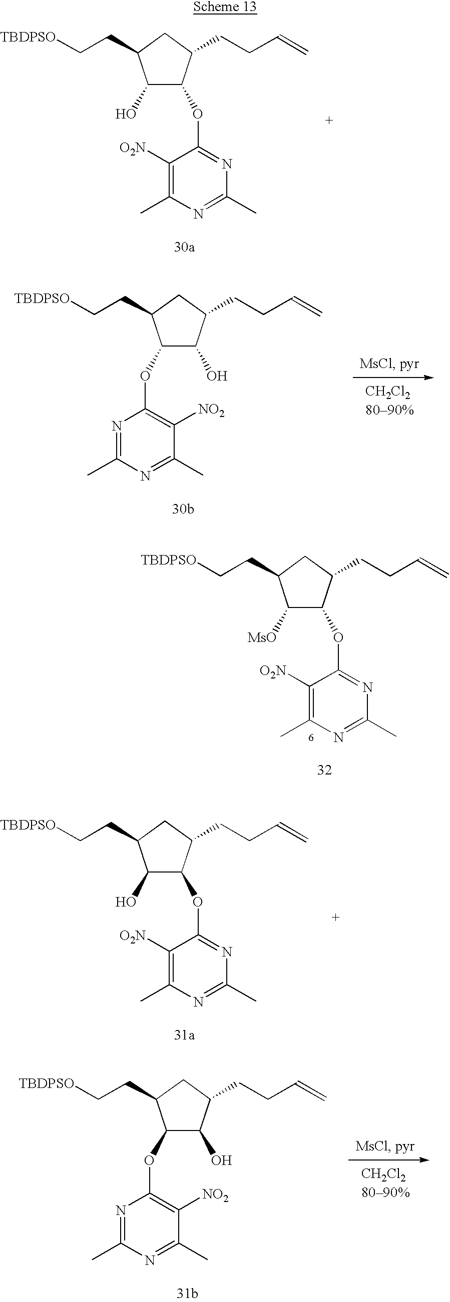

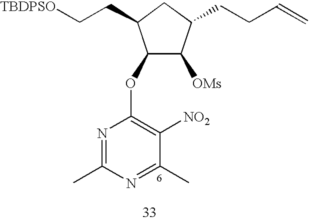

This phenomenon can be explained in two ways. In the first case, the electrophile (MsCl) attacks the Meissenheimer zwitterion from the least sterically demanding side as shown in Scheme 14.

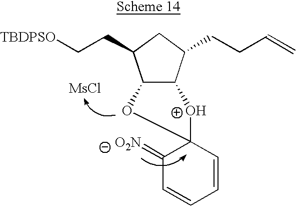

Alternatively, the mesylation of 30a (31b) may be simply faster than that of 30b (31a) as shown in Scheme 15.

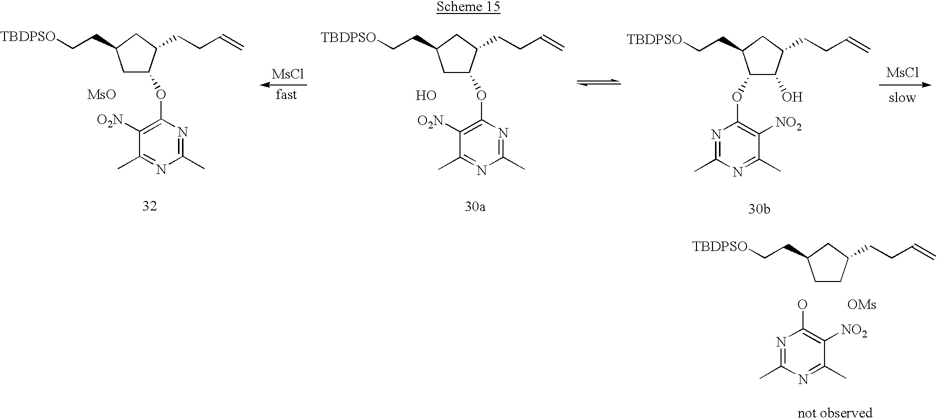

It should be noted that compound 32, after reduction of the nitro group and an S_{N}2 ring closure, would provide the morpholino system with the incorrect trans stereochemistry as well as with the regiochemistry opposite to that which is required in peptidomimetic molecule (vi) (methyl at C6 should be on the same side of the molecule as the olefin) (Scheme 13). However, closure of the reduced form of mesylate 33 would provide the correct regiochemical orientation, but with the stereochemistry in the trans fused ring being that of peptidomimetic molecule (xxii). As determined in the computational analysis of Example 1 above, the tricycle scaffold structure derived from 33 should function well as a mimetic (good overlap of the pharmacophores is maintained) for both molecule (vi) and molecule (xxii), since only a small “tilt” in the core tricyclic ring system results from the different stereochemistry. Further chemical transformations were performed on 32 as a model for 33.

The next, essential step before S_{N}2 ring closure was reduction of the 5-nitro group to the amine. Dozens of methods exist for reducing aromatic nitro groups, so it was very frustrating and surprising that this step proved to be so problematic. Problems may arise in the handling of an a arylamine, prepared by reduction of the corresponding nitroarene, because of rapid oxidation by exposure to air (De Riccardis, F.; Bonala, R. R.; Johnson, F. J. Am. Chem. Soc. 1999, 45, 10453-10460). Treatment of 32 or 33 with Pd/C or PdCl₂ (60 psi, EtOH/THF) or with Pd/C and ammonium formate in MeOH, all affected the desired transformation, yet the yields were not reproducible or scalable. Treatment with iron(III) and hydrazine-hydrate gave no reaction (Hine, J.; Hahn, S.; Miles, D. E.; Ahn, K. J. Org. Chem. 1985, 50, 5092-5096). Zinc or iron powder in acetic acid and ethanol looked promising by TLC, but the reaction was not completely clean. Unfortunately, nearly all of the compounds described in this synthesis do not withstand silica gel purification. Many specific examples for reducing the 5-nitro pyrimidine of pteridines or thiopterins indicated using either stannous chloride (SnCl₂) (Agrofolio, L. A.; Demaison, C.; Toupet, L. Tetrahedron 1999, 55, 8075-8083; Gourdel-Martin, M. E.; Huet, F. J. Org. Chem. 1997, 62, 2166-2172) in ethanol or sodium dithionite (Na₂S₂O₄) in DMF-H₂O (Nair, M. G.; Boyce, L. H.; Berry, M. A. J. Org. Chem. 1981, 46, 3354-3357. b) Taylor, E. C.; Barton, J. W.; Paudler, W. W. J. Am. Chem. Soc. 1961, 83, 4961-4967. c) Pendergast, W.; Hall, W. R. J. Heterocyclic Chem. 1986, 23, 1411-1413. d) Haddow, J.; Suckling, C. J.; Wood, H. C. S. J. Chem. Soc., Perkin Trans. 1 1989, 1297-1304). While Na₂S₂O₄ proved difficult to use on small scale, SnCl₂ rendered clean product, albeit in moderate and unpredictable yields (30-50%). The work up, treatment of the ethanolic reaction mixture with aqueous NaHCO₃ and EtOAc, caused an intractable solid to form from which it was very difficult to isolate the product. Analysis of the aqueous layer revealed desilylated, reduced product and ethyl tert-butyldiphenyl ether. The silyl ethyl ether was further characterized by ¹H NMR. Because of this side reaction, the pivoyl protected analogs 34a/b and 35a/b were generated (not shown) by selectively protecting the primary alcohols 9a and 9b (PivCl, DMAP, pyridine). Coupling to pyrimidine 29 generated products 36a and 36b and diastereomers 37a and 37b (Scheme 16).

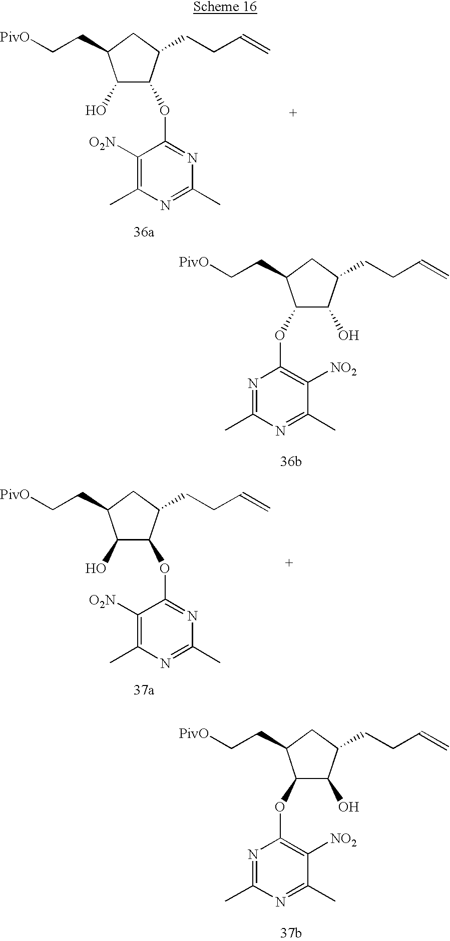

Mesylation of 36a/b gave compound 38, which was subjected to the SnCl₂ conditions. Fortunately, the desired amine 40 was reproducibly isolated in 70-80% yield (Scheme 17). Mesylation of 37a/b also formed only a single isomer 39 (not shown).

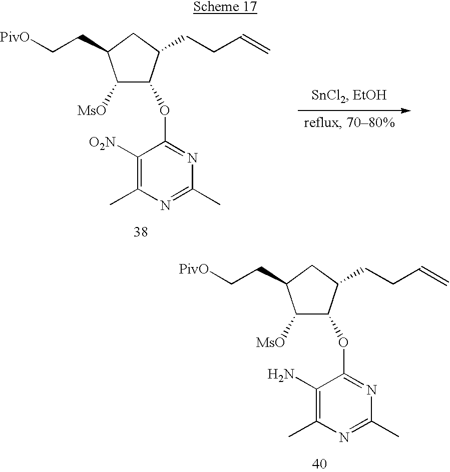

The direct ring-closure of 40 to 41 shown in Scheme 18 was attempted using a variety of solvents and conditions. The polar, aprotic solvents, DMF and acetonitrile, were used in conjunction with pyridine or K₂CO₃ at 60° C. and 90° C., respectively, but no reaction occurred. Protic solvents were also tried to determine whether an S_{N}1-type reaction could be induced. Dioxane-H₂O at 85° C. for 20 hrs, DME-H₂O-LiCl at 95° C. for 24 hrs, and n-butanol/K₂CO₃ at 117° C. were all unsuccessful and left the starting material unchanged. Therefore, 40 was treated with TosCl and pyridine in CH₂Cl₂ to generate the sulfonamide 42 (50%), which was in turn deprotonated with various bases in hope that the corresponding anion would undergo the desired S_{N}2 reaction. In addition to the toluene sulfonamide, the allyloxycarbamate (allylchloroformate, pyridine, 74%) and formamide (formic acid, EDCI, NMM, 50%) derivatives were examined. Bases investigated were LDA, NaH, and KOBu^{t}. Only one set of conditions generated a new spot by TLC. The sulfonamide derivative, when treated with 2.0 eq. of KOBu^{t} in THF under reflux produced the elimination product 43 in poor yield. Starting material was also recovered, but total mass recovery was very poor. Compound 43 is isomeric with the desired tricycle 41. The structure of 43 was assigned based on D₂O quenching of the NH proton in CDCl₃, and the new vinyl peaks in both the proton and carbon NMR results.

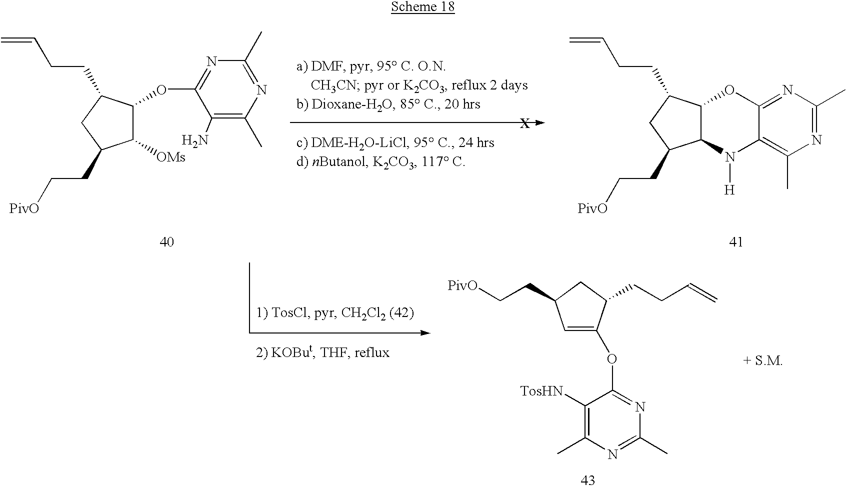

In order to test other good leaving groups in the S^{N}2 reaction, the tosylate and triflate analogs of 38 were prepared. However, the tosylation reaction resulted in poor conversion and did not facilitate coupling and the triflate compound did not survive the SnCl₂ reduction.

Although it is possible that the intramolecular reaction did not proceed due to a poor trajectory for the nucleophilic substitution, an intermolecular reaction between 40 and sodium azide to produce 44 was equally unreactive (Scheme 19). Therefore, we decided that the sp³ center in this particular molecule was simply too unreactive to be of synthetic use.

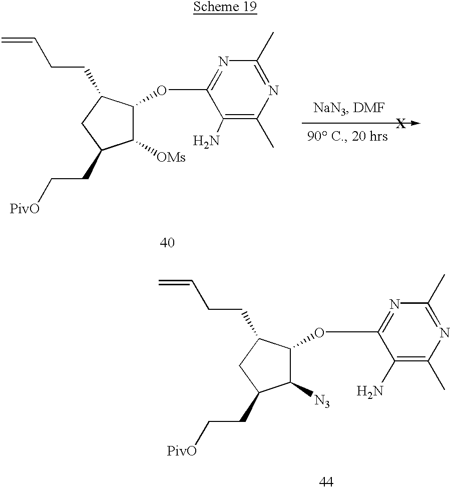

Since the secondary mesylate 40 was unreactive toward nucleophiles, it was conceived that ring closing by a reductive amination reaction would be more productive. First, the sidechain olefin of 36a and 36b was elaborated to a protected alcohol (Scheme 20) in order to reduce the number of transformations needed after the tricyclic core was generated. Hence, the regioisomeric mixture 36a and 36b was dihydroxylated with AD-Mix- [symbol] to produce the corresponding diols 45a/b (not shown), oxidatively cleaved (NaIO₄) to the corresponding aldhehydes 46a/b (also not shown), and reduced (NaBH₄) to the alcohols 47a and 47b. No purification was performed throughout the three-step sequence of Scheme 20.

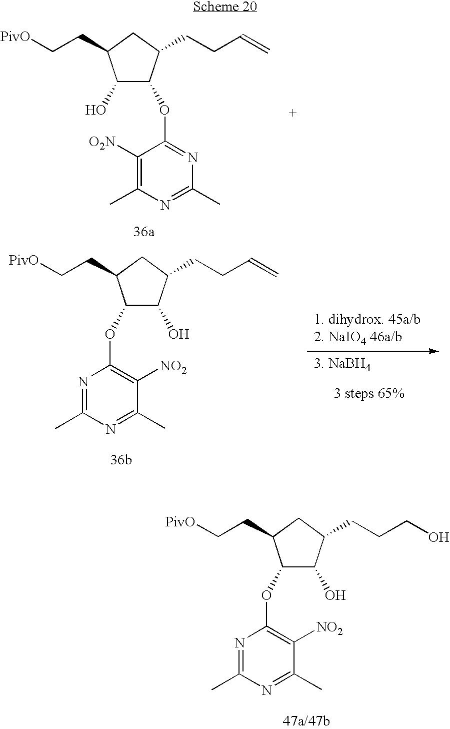

Interestingly, the primary alcohols after silyl protection (48a/b) using TBDPSCl/ DMAP (Scheme 21) underwent Smiles rearrangement much slower in comparison to the intermediates 45a/b-46a/b. The same result was obtained with the —OTBDPS diastereomers 49a/b derived from 37a/b. Therefore, the regioisomers were separated by chromatography and oxidized to the corresponding ketone.

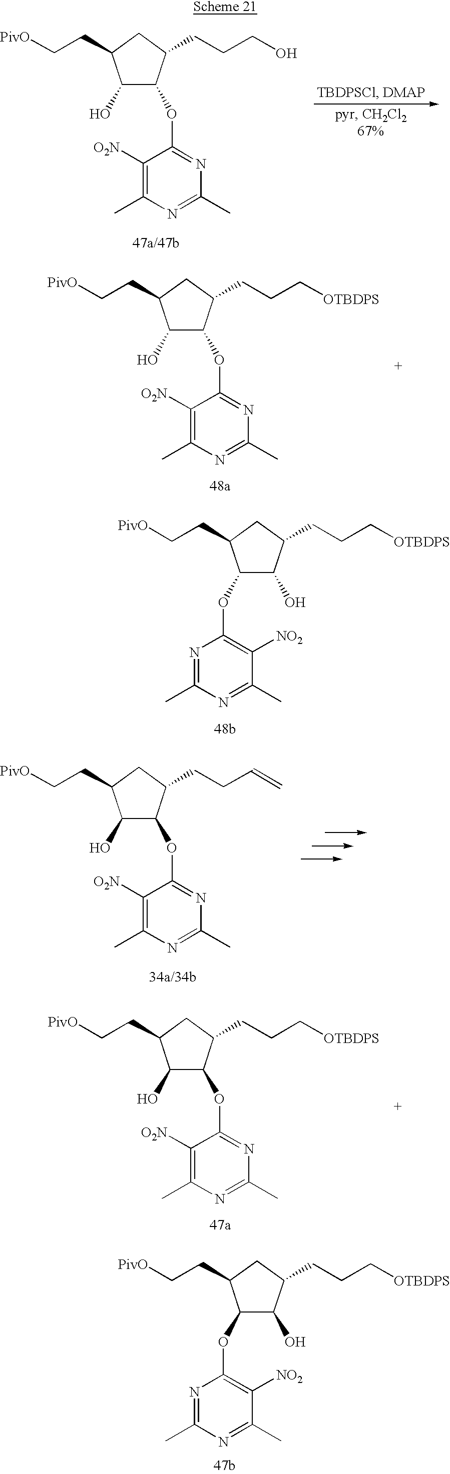

The ratio of 48a to 48b was ˜2:1 by ¹H NMR integration, while the ratio of 49a to 49b was ˜1.3:1. It was determined by NMR (TOCSY (Crews, P.; Rodriguez, J.; Jaspars, M. Organic Structure Analysis; Houk, K. N., Ed.; Oxford University Press: New York, 1998; pp 181-204), NOE) of the corresponding ketones 50a and 50b (Scheme 22), and 51a and 51b (not shown) that the major isomer in each case was the undesired regiomeric product 50a or 51a. The wrong isomers (48a, 49a) could be reisomerized back to the original ratio by heating in CH₂Cl₂ (40° C., 4 days) or toluene (80° C., ˜12 hours) and the desired compounds could again be isolated by chromatography.

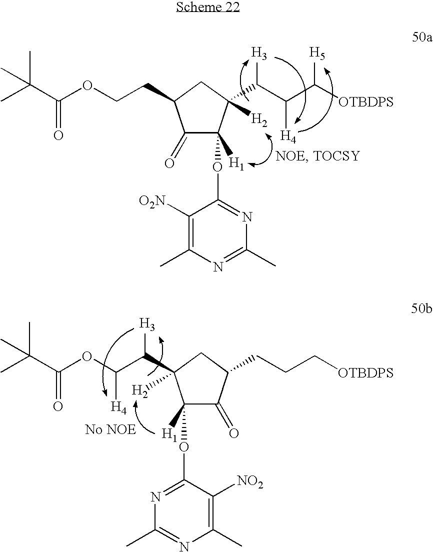

The oxidation of the secondary alcohol needed to be fast so that the product ketones would be formed before rearrangement occurred. Thus, some development was required. Swern conditions degraded the molecule, and oxidation by TPAP/NMO (Griffith, W. P.; Ley, S. V. Aldrichimica Acta 1990, 23, 13-19) only worked well for some diastereomers (There are 4 isomers: a set of diastereomers, and a set of regioisomers for each diastereomer). The best and fastest set of conditions for all of the isomers was a TEMPO and bleach oxidation (De Nooy, A. E. J.; Besemer, A. C.; van Bekkum, H. Synthesis 1996, 1153-1124). The desired ketones (50b, 51b) were obtained in minutes without evidence of isomerization as shown in Scheme 23. The incorrect regioisomeric ketone 50a was used as a model in subsequent steps (Scheme 24 below).

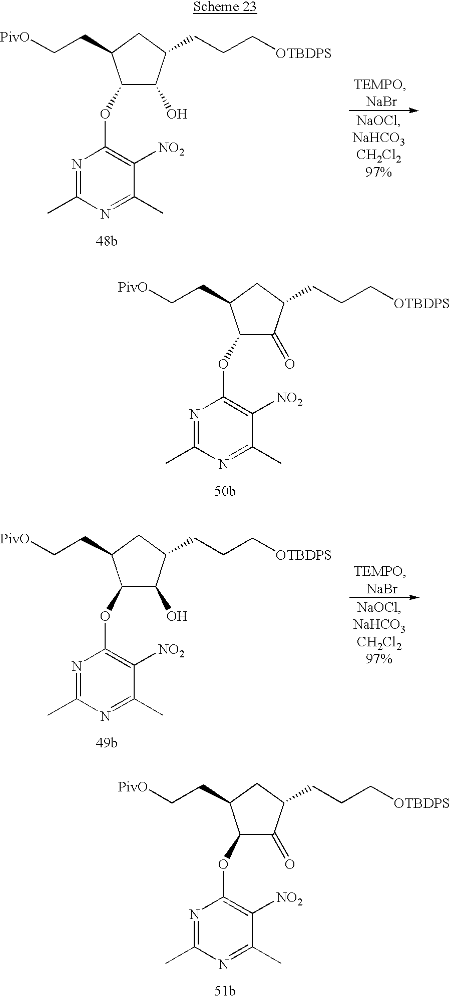

Reduction of the nitro group,was required next so that the amination reaction could proceed. The SnCl₂ conditions used previously (Scheme 17) had to be avoided since the silyl protection group was again being used. Initially, Raney nickel (60 psi, EtOH) appeared promising, but over time the results became irreproducible. Conditions employing commercial Raney nickel (pH>9.5) resulted in reduction of the nitro group and concomitant formation of imine 52 as shown in Scheme 24. A small amount of amine 53 was also generated. Neutral Raney nickel, made as described in Organic Synthesis, Collective Volume III; Horning, E. C., Ed.; John Wiley & Sons: New York, 1955; pp 181-183, formed imine 52 and its precursor, hemiaminal 54 (Scheme 24). The hemiaminal decomposed to the imine upon sitting over night. Again, however, the nickel conditions were not reproducible.

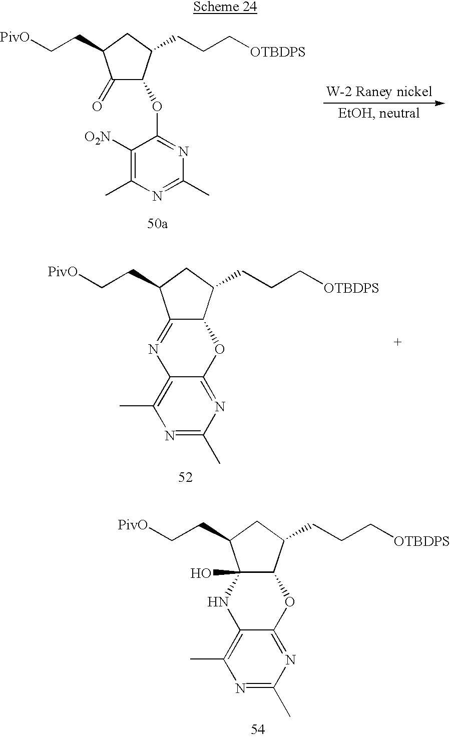

Reduction of 50b using either iron or zinc powder in acetic acid (10 eq.) and ethanol resulted in clean conversion (90%) to imine 55 (or from 50a to 52), as shown in Scheme 25.

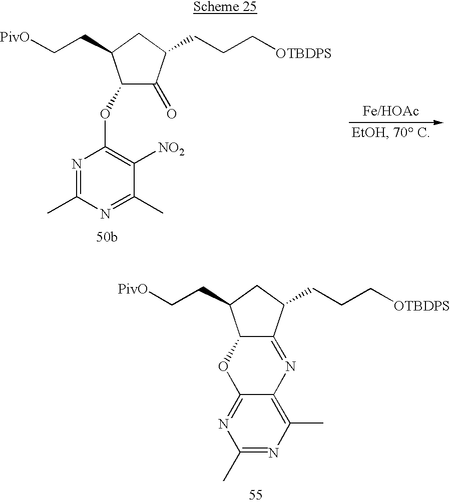

It was initially believed that imine 55 could be stereoselectively reduced from the bottom face to generate the target trans fused system. However, reduction with NaBH₄ in MeOH or NaBH₃CN (HCl, MeOH) produced only the cis fused isomer 56, as determined by NOE, in good yield (75%) as shown in Scheme 26. Reduction of 52 also yielded the cis fused isomer 53 (not shown).

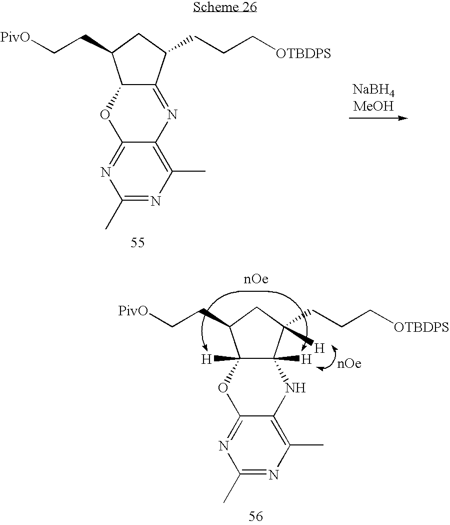

Reducing agents that could coordinate to a heteroatom were expected to deliver a hydride from the same face as the ether oxygen to provide the trans fused system (Hoveyda, A. H.; Evans, D. A.; Fu, G. C. Chem. Rev. 1993, 93, 1307-1370). However, reduction of imine 52 with lithium aluminum hydride (1.0 eq., THF) (Cawley, J. J.; Petrocine, D. V. J. Org Chem. 1976, 41, 2608-2611) or DIBAL (2.0 eq., CH₂Cl₂, −78° C.) (McGrane, P. L.; Livinghouse, T. J. Org. Chem. 1992, 57, 1323-1324) only resulted in deprotection of the pivoyl group followed by either a ring formation resulting from the liberated primary alcohol closing onto the imine (57a), or rearrangement to the enamine (57b) in the manner shown in Scheme 27. It was determined that the amine was present because of a characteristic purple color that is evident with UV irradiation on TLC plates. Compounds 57a and 57b are structural isomers and thus have the same mass. The NMR's of the two compounds were expected to be significantly different in the areas indicated in Scheme 27, and IR spectroscopy specified an alcohol. Therefore, 57b has been assigned the product of 52 after DIBAL treatment. Reduction of enamine 57b has not been attempted, but it is expected that hydrogen would add from the top face for the same reason (sterics) that NaBH₄ or nickel added to the top face of imines 52 and 55.

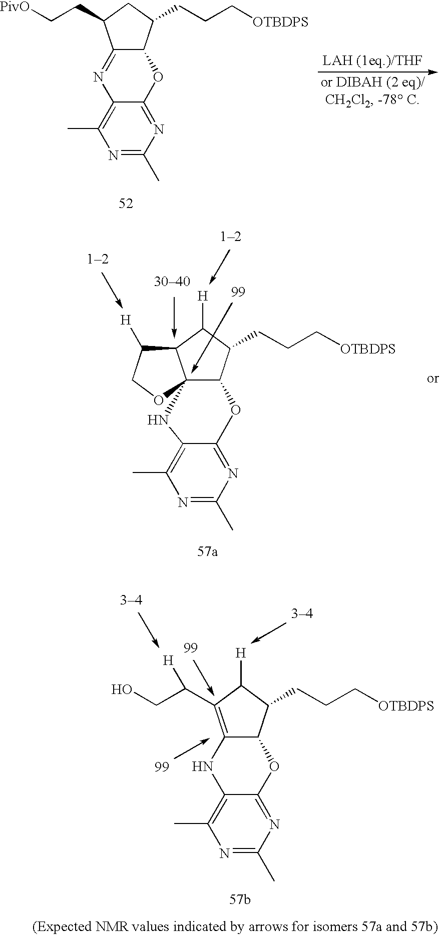

Other coordinating reducing agents that either gave no reaction or a messy reaction include zinc borohydride [Zn(BH₄)₂] (Cimarelli, C.; Palmieri, G. Tetrahedron: Asymmetry 2000, 11, 2555-2563, and refs. therein; Fustero, S.; Pina, B.; de la Torre, M. G.; Navarro, A.; de Arellano, C. R.; Simon, A. Org. Lett. 1999, 1, 977-980), dimethylamine borane, lithium aminoborohydride (LiMe₂NBH₃), and borane methylsulfide (BMS) (Singaram, B.; Goralski, C. T. Transition Metals in Organic Synthesis; Beller, M.; Bolm, C., Ed.; Wiley-VCH Verlag GmbH: Weinheim, Germany, 1998). Reducing metal conditions (Na, NH₃, ether) were expected to furnish predominately the most thermodynamically favored product, which has not been determined for this molecule, but did not give a clean reaction (Smith, M. B. Organic Synthesis; McGraw-Hill, 1994; pp 459; Bhattacharya, S.; Mandal, A. N.; Raychaudhuri, S. R.; Chatterjee, A. J. Chem. Soc., Perkin Trans. 1 1984, 5-13; Maiti, S. B.; Raychaudhuri, S. R.; Chatterjee, A. J. Chem. Soc., Perkin Trans. 1 1988, 3, 611-621).

In comparison, the desired regioisomeric imine 55 was treated with LAH (THF, −78° C.) as shown in Scheme 28. The first equivalent caused a fast deprotection of the pivoyl group, but the imine remained intact (58). A second equivalent resulted in reduction of the imine to the cis product 59, determined by comparison of ¹H NMR shifts to the pivoyl protected compound and by NOE experiments.

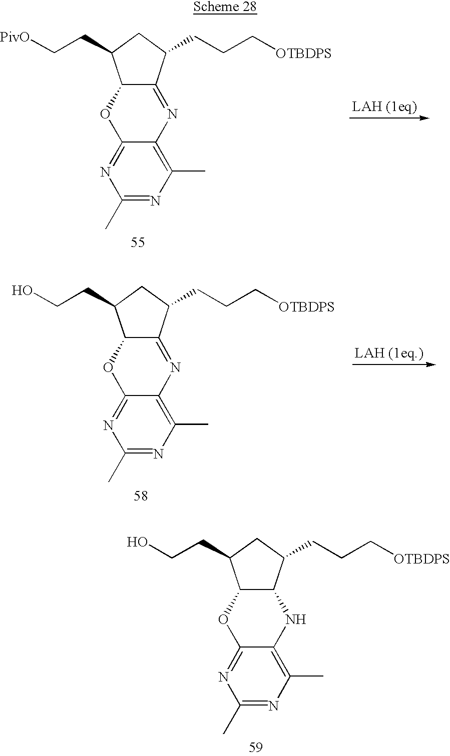

The imines 52 and 55, enamine 57b, and the cis product 59 all have the tricyclic skeleton or scaffold of compounds (ii) and (iii) above. The trans variation in the scaffold structure, as occurs in molecules (vi) and (xxii), were not obtained by the above procedures.

The cis compound 59, which provides the basic scaffold structure of compounds (ii) and (iii), may be functionalized by conventional techniques used in peptide chemistry to provide the Arg-, Tyr-, Ile- and Ser- groups in the desired locations. As shown in Scheme 29, treatment of 59 with two equivalents of lithium diisopropylamide (LDA) in THF, followed by addition of 4-benzylOTBDPS provides compound 60.

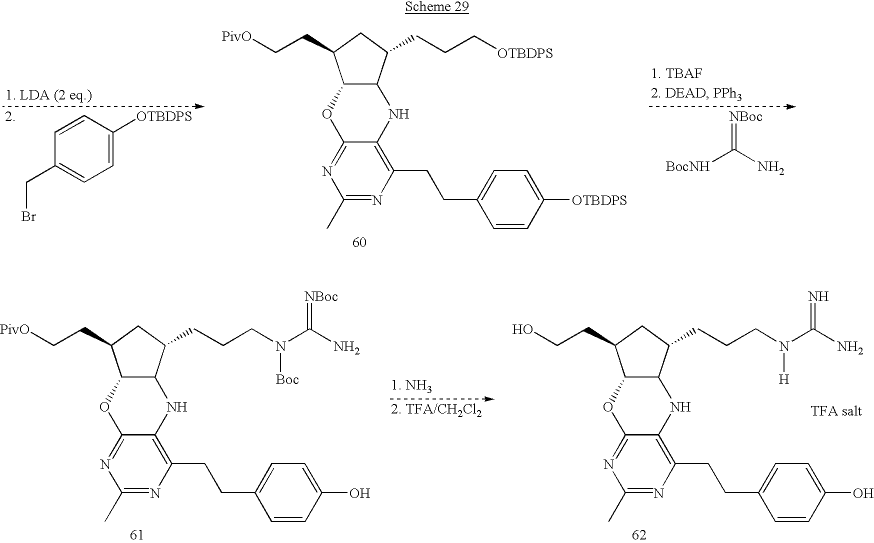

The TBDPS groups are cleaved from compound 60 by treatment with tetrabutylammonium fluoride (TBAF) in THF, followed by reaction with the protected amine BocN=C(NHBoc)(NH₂) precursor for arginine using the Mitsunobu reaction (Mitsunobu, et al., Bull. Chem. Soc. Jpn., 1967, 40, 2380; Synthesis, 1981, 1) with triphenylphosphane and diethyl azodicarboxylate (DEAD) followed by hydrazinolysis to convert the aliphatic hydroxyl group to the tertiary amine 61 (phthalimide is not required in this case). The TBAF also deprotects the aromatic hydroxyl group to provide a tyrosine-like group in the desired position on the scaffold portion of the molecule.

Compound 61 is treated with aqueous ammonia to remove the pivoloyl group and deprotect the aliphatic hydroxyl group to provide a serine-like group. The resulting compound is treated with trifluoroacetic acid in methylene chloride in a conventional manner to remove the Boc- groups to provide an arginine-like group in compound 62 as a trifluoroacetic acid salt. Compound 62 is a representative or subset compound (xxv), noted above. In the specific case of compound 62, the isoleucine-like group is provided in the form of a simple methyl group.

The details of the synthesis of the various individual compounds from the above schemes are as follows:

Compound 9a

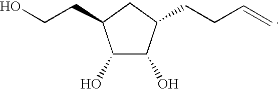

A solution of diene 8 (6.60 g, 39.8 mmol) and methanesulfonamide (3.78 g, 39.8 mmol) in 300 mL t-BuOH—H₂O 1/1 was treated with 59.64 g of AD-mix- [symbol] (Aldrich) (1.5 g/mmol, olefin) over 15 min. The mixture was allowed to react for 5 h at room temperature and was filtered. The solids were washed with t-BuOH/AcOEt 1/3 (3×50 mL) and discarded. The whole liquid parts were combined and the aqueous phase was separated and extracted with more ethyl acetate (2×50 mL). All organic phases were combined and evaporated. The residue was redissolved in 200 mL of ethyl acetate and the aqueous phase was separated and extracted with ethyl acetate (1×50 mL). The organic phases were combined, dried over magnesium sulfate and concentrated under reduced pressure to afford 5.30 g of a mixture of diastereoisomeric triols 9a and 9b (approximately 1/1) and a small amount of methanesulfonamide. The isomers were separated by chromatography on SiO₂ (hexanes/EtOAc, 1:10) to afford 1.925 g of triol 9a (9.6 mmol, 24%), 0.620 g of mixed isomers and 1.050 g of triol 9b. Triol 9a ¹H NMR (500 MHz, CDCl₃) δ1.41 (m, 2H), 1.64 (m, 4H), 1.86 (m, 1H), 2.05 (m, 3H), 3.41 (br s, 1H), 3.59 (m, 2H), 3.74 (m, 1H), 3.90 (s, 1H), 4.16 (br s, 1H), 4.75 (br s, 1H), 4.93 (d, J=10.5 Hz, 1H), 4.96 (dd, J=1.5 Hz, J=17 Hz, 1H), 5.78 (m, 1H); ¹³C (62.5 MHz, CDCl₃) δ29.3, 32.3, 34.6, 37.7, 40.0, 40.6, 62.1, 75.4, 80.8, 114.6, 139.0 IR (film) cm⁻¹: 3374, 2931, 1640. HRMS Calcd for [M+1]⁺ CHO:; found

Compound 9b

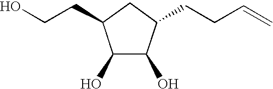

¹H NMR (500 MHz, CDCl₃) δ1.36 (m, 2H), 1.70, (m, 3H), 1.83 (m, 1H), 2.0-2.76 (m, 4H), 2.76 (br s, 1H), 2.82 (br s, 1H), 3.50 (br s, 1H), 3.63 (m, 2H), 3.76 (m, 1H), 3.99 (m, 1H), 4.93 (d, J=10 Hz, 1H), 4.99 (d, J=21 Hz, 1H), 5.82 (m, 1H); ¹³C (62.5 MHz, CDCl₃) δ32.6, 32.9, 33.8, 38.2, 42.3, 61.7, 75.1, 80.5, 114.5, 139.0. IR (film) cm⁻: 3360, 2929, 1639. Anal. Calcd. for C₁₁H₂₀O₃: C, 65.97; H, 10.07. Found: C, 65.79; H, 10.18.

Compound 34a

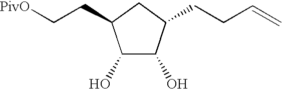

A cooled (0° C.) solution of triol 9a (4.49 g, 0.022 mol), pyridine 2.9 mL, 0.023 mol) and DMAP (0.134 g, 0.0011 mmol) in dry CH₂Cl₂ (11 mL) was treated with a solution of pivoyl chloride in CH₂Cl₂ (11 mL) via addition funnel. The reaction was allowed to come to room temperature and after 3 h the reaction mixture was washed with 10% HCl. The aqueous layer was back extracted with CH₂Cl₂. The combined organics are washed with brine, dried (MgSO₄) and evaporated to an oil. The crude oil is column purified using silica gel (10:1) to remove traces of bis-pivoylated material and starting material, affording 4.24 g (67%) of the desired material. ¹H (500 MHz, CDCl₃) δ1.16 (s, 9H), 1.40 (m, 2H), 1.65 (m, 3H), 1.88 (m, 2H), 1.95 (m, 1H), 2.02 (q, J=7.5 Hz, J=14.5 Hz), 2.71 (br s, 1H), 3.04 (br s, 1H), 4.90 (dd, J=1 Hz, J=10.5 Hz), 4.96 (dd, J=1.5 Hz, J=15 Hz), 5.77 (m, 1H); ¹³C (62.5 MHz, CDCl₃) δ27.3, 29.1, 32.3, 34.0, 34.2, 38.9, 39.6, 40.2, 63.8, 75.1, 80.6, 114.6, 138.9, 179.0. IR (neat oil) cm⁻¹: 3429, 1727, 1640. Anal. Calcd. for C₁₆H₂₈O₄: C, 67.57; H, 9.92. Found: C, 67.70; H, 10.07.

Compound 34b

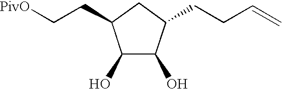

¹H (500 MHz, CDCl₃) δ1.17 (s, 3H), 1.36 (m, 2H), 1.68 (m, 3H), 1.92 (m, 3H), 2.10 (m, 2H), 2.86 (br s, 1H), 3.62 (dd, J=4 Hz, J=8.5 Hz, 1H), 3.90 (t, J=3.5 Hz, 1H), 4.91 (d, J=10.5 Hz, 1H), 4.97 (s, J=17 Hz, 1H), 5.79 (m, 1H); ¹³C (62.5 MHz, CDCl₃) δ27.3, 29.1, 32.5, 33.8, 34.2, 37.2, 38.9, 42.6 63.7, 75.2, 80.6, 114.6, 138.9, 178.9. IR (neat oil) cm⁻¹: 3429, 1727, 1640. Anal. Calcd. for C₁₆H₂₈O₄: C, 67.57; H, 9.92. Found: C, 67.35; H, 10.03.

Compounds 36a and 36b

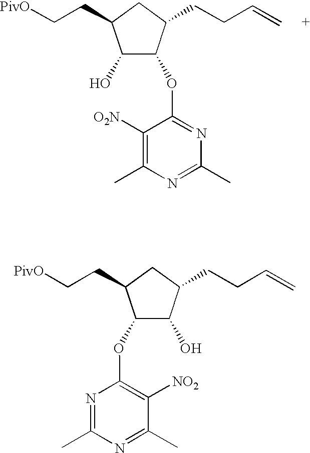

A suspension of cyclopentane diol 34a (1.51 g, 3.45 mmol) and Bu₂SnO (0.91 g, 3.62 mmol) in 25 mL of methanol was heated to reflux for 6 hours or until the solid white Bu₂SnO disappeared. The mixture was concentrated under reduced pressure, redissolved in dry THF, concentrated again,and dryed on an oil pump. The resulting oil was dissolved in 40 mL of dry THF and 1.16 g of tetrabutylammonium bromide (TBAB) was added, followed by a solution of 0.760 g (4.03 mmol) of 4-chloro-5-nitro-2,6-dimethylpyrimidine (29) in THF (1 mL). The mixture was allowed to react for ˜24 h at reflux temperature. After this time, the mixture was concentrated under reduced pressure and the crude material was purified by chromatography on SiO₂ (50:1)(hexanes/EtOAc, 3:1) to afford a 1:1 mixture of regiocoupled products (1.61 g, 78% yield). ¹H (500 MHz, CDCl₃) (mixture of regioisomers) δ0.9 (t, J=7.5 Hz, 2H), 1.15 (s, 9H), 1.19 (s, 1H), 1.23 (m, 2H), 1.34-2.18 (m, 15H), 2.46, (m, 2H), 2.49 (s, 6H), 2.59 (s, 3H), 2.60 (s, 3H), 3.85 (m, 1H), 4.08 (m, 2H), 4.13 (m, 2H), 4.25 (t, J=3.5 Hz, 1H), 4.98 (m, 4H), 5.21 (dd, J=8.5 Hz, J=4 Hz 1H), 5.71 (m, 1H), 5.71 (m, 1H), 5.80 (m, 1H); ¹³C (125 MHz, CDCl₃) δ13.5, 20.5, 26.1, 26.2, 26.9, 27.22, 27.28, 28.5, 29.1, 32.0, 32.1, 32.8, 33.1, 33.3, 33.4, 37.0, 38.2, 38.8, 39.4, 40.3, 62.9, 63.3, 72.8, 80.2, 82.9, 85.3, 114.7, 115.0, 138.2, 138.7, 160.1, 160.62, 160.65, 161.3, 168.3, 168.5, 178.6, 178.7. IR (film) cm⁻¹: 3430, 1726, 1640. HRMS Calcd for [M+1]⁺ C₂₂H₃₃N₃O₆: calc 436.2447; found 436.2449.

Compounds 37a and 37b

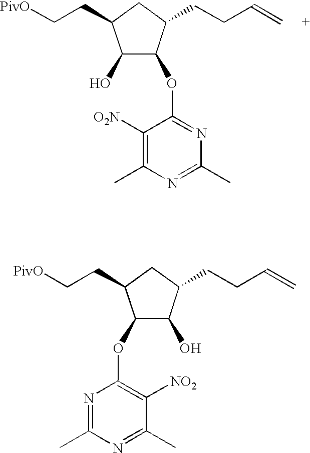

¹H (500 MHz, CDCl₃) (mixture of regioisomers) δ0.88 (m, 1H), 1.18 (s, 9H), 1.20 (s, 9H), 1.20 (m, 3H), 1.42-2.14, (m, 16H), 2.46 (m, 1H), 2.39(m, 1H), 2.51 (s, 3H), 2.52 (s, 1H), 2.615 (s, 3H), 2.619 (s, 3H), 3.87 (dd, J=3.5 Hz, J=8.5 Hz, 1H), 4.06 (m, 2H), 4.12 (m, 2H), 4.28 (t, J=3.5 Hz, 1H), 4.98 (m, 4H), 5.19 (dd, J=3.5 Hz, J=8.5 Hz; 1H), 5.69 (t, J=3.5 Hz), 5.80 (m, 2H); ¹³C (125 MHz, CDCl₃) δ20.5, 20.6, 26.1, 27.2, 28.5, 29.0, 29.1, 32.0, 32.3, 32.5, 32.9, 33.5, 33.6, 33.8, 33.9, 34.1, 36.0, 37.1, 37.2, 39.4, 42.8, 62.9, 63.4, 63.6, 73.0, 75.1, 80.4, 80.6, 83.3, 85.4, 114.7, 114.8, 138.3, 138.6, 138.8, 160.2, 160.5, 160.8, 161.3, 168.4, 168.5, 178.6, 178.7 IR (film) cm⁻¹: 3489, 1726, 1640. HRMS Calcd for [M+1]⁺ C₂₂H33N₃O₆: calc 436.2447; found 436.2449.

Compound 38

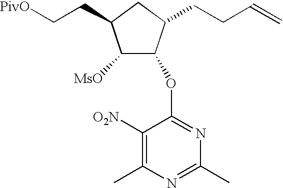

A solution of alcohols 36a/36b (1.0 g, 2.28 mmol) in CH₂Cl₂ (5 mL) was added to a solution of MsCl (0.353 mL, 4.56 mmol) and pyridine (0.402 mL, 5.01 mmol) in 5 mL CH₂Cl₂. The reaction was allowed to stir 20 h. The solvent was removed and the pyridinium salts were removed by trituration with 1:1 EtOAc/Hex. Any remaining MsCl was removed by heating to 60° C. under high vacuum. The crude yield, 939 mg (80.2%) was utilized without purification in the next step. ¹H (500 MHz, CDCl₃) δ1.20 (s, 9H), 1.41 (m, 1H), 1.56 (m, 1H), 1.63 (m, 2H), 1.77 (m, 2H), 2.02 (m, 2H), 2.21 (m, 1H), 2.43 (m, 1H), 2.50 (s, 3H), 2.60 (s, 3H), 2.93 (s, 3H), 4.10 (m, 2H), 4.67 (dd, J=3.5 Hz, J=9 Hz, 1H), 4.94 (m, 2H), 5.69 (m, 1H), 6.00 (t, J=3.5 Hz, 1H); ¹³C (62.5 MHz, CDCl₃) δ20.1, 25.7, 27.0, 28.7, 31.6, 31.8, 31.9, 37.2, 37.9, 38.2, 38.6, 38.7, 62.4, 78.6, 84.0, 115.1, 132.1, 137.6, 160.14, 160.19, 167.9, 178.2. IR (film) 2960, 2937, 2873, 1724, 1640. HRMS Calcd for [M+1]⁺C₂₃H₃₅N₃O₈S: calc 513.2144; found 514.2225.

Compound 39

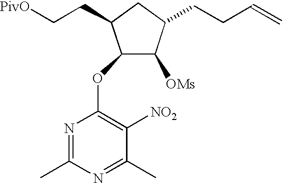

¹H (500 MHz, CDCl₃) δ1.18 (s, 9H), 1.20, (m, 3H), 1.38-2.40 (m, 7H), 2.52 (s, 3H), 2.62 (s, 3H), 2.93 (s, 3H), 4.05 (t, J=6.5 Hz, 2H), 4.69 (dd, J=3.5 Hz, J=9Hz, 1H), 5.00 (m, 2H), 5.79 (m, 1H), 5.98 (t, J=4 Hz, 1H); ¹³C (125 MHz, CDCl₃) δ20.3, 25.8, 27.1, 28.8, 31.7, 31.9, 32.6, 35.8, 38.3, 39.7, 62.4, 78.8, 84.2, 115.1, 126.9, 137.6, 160.0, 160.2, 168.2, 178.3. IR (film) cm⁻¹: 1725. HRMS Calcd for [M+1]⁺ C₂₃H₃₅N₃O₈S: calc 514.2223; found 514.2225.

Compound 40

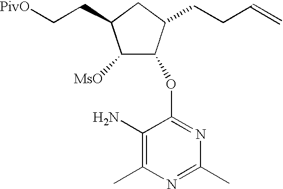

The nitro mesylate 38 (200 mg, 0.387 mmol) was heated with SnCl₂ (294 mg, 1.55 mmol) in 2 mL EtOH at 70° C. for 1.5 h. The warm reaction mixture was poured into 40 mL crushed ice and neutralized with sat. aq. NaHCO₃. The solids were isolated by filtration and stirred with EtOAc. The water layer was extracted several times with EtOAc. The combined organics were dryed (MgSO₄) and concentrated to yield 140 mg, 71.8%. This compound was used without further purification in the next step. ¹H (500 MHz, CDCl₃) δ1.20 (s, 9H), 1.37 (m, 1H), 1.55 (m, 1H), 1.67 (m, 2H), 1.85 (m, 1H), 2.04 (m, 3H), 2.23 (m, 1H), 2.32 (s, 3H), 2.44 (m, 1H), 2.88 (s, 3H), 3.49 (br s, 2H), 4.12 (m, 2H), 4.74 (dd, J=4 Hz, J=9 Hz, 1H), 4.92 (m, 2H), 5.68 (m, 1H), 5.91 (t, J=4.5 Hz, 1H); ¹³C (125 MHz, CDCl₃) δ19.2, 24.9, 27.3, 29.1, 31.8, 32.3, 32.5, 38.2, 38.4, 38.8, 38.9, 63.0, 75.6, 85.6, 115.2, 123.5, 138.1, 148.9, 155.3, 156.9, 178.7. IR (film) cm⁻¹: 3374, 1725.

Compound 42

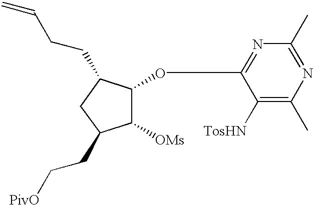

Amino mesylate 39 (288 mg, 0.592 mmol) was dissolved in 3 mL of dry CH₃CN. Solid TosCl (0.124 mL, 0.651 mmol) was added followed by pyridine (0.062 mL, 0.769 mmol). The reaction did not go to completion despite addition of more pyridine and TosCl (0.3 eq. each). After 24 h, the mixture was evaporated and triturated with 1:1 EtOAc/Hex to remove the pyridinium salts. The crude material, 347 mg (91.6%) was used in the next step. ¹H (500 MHz, CDCl₃) δ1.05 (m, 1H), 1.22 (s, 9H), 1.30 (m, 1H), 1.50 (m, 1H), 1.58 (m, 1H), 1.65 (m, 1H), 1.95 (m, 3H), 2.10 (m, 1H), 2.37 (m, 1H), 2.39 (s, 3H), 2.41 (s, 3H), 2.54 (s, 3H), 2.91 (s, 3H), 4.10 (m, 2H), 4.59 (dd, J=4.5 Hz, J=9 Hz, 1H), 4.95 (m, 2H), 5.68 (m, 2H), 6.22 (br s, NH), 7.26 (d, J=8.5 Hz, 2H), 7.66 (d, J=8.5 Hz, 2H); ¹³C (62.5 MHz, CDCl₃) δ21.0, 21.5, 25.4, 27.1, 28.2, 31.6, 31.8, 32.3, 37.9, 38.4, 38.7, 62.6, 84.7, 99.1, 114.0, 115.0, 127.2, 129.8, 137.5, 137.8, 143.9, 164.5, 164.9, 166.4, 178.4. IR (film) cm⁻¹: 3256, 1723, 1640. HRMS Calcd for [M+1]⁺ C₃₀H₄₃N₃O₈S₂: calc 638.2569; found 638.2570.

Compound 43

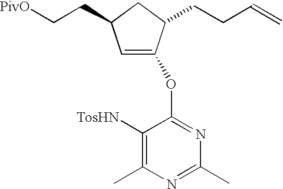

Compound 42 (˜340 mg, 0.531 mmol) was treated with ^{t}BuOK (59.6 mg, 1.062 mmol) in dry THF at reflux temperature. After 19 h, a new spot (TLC) was observed, but the reaction did not go to completion even after 3 days. The mixture was quenched with 10% HCl and extracted with EtOAc. Column purification produced 45 mg of the title compound and 27 mg of s.m. ¹H (500 MHz, CDCl₃) δ1.18 (s, 9H), 1.14 (m, 1H), 1.31 (m, 1H), 1.61-1.72 (m, 3H), 1,86 (m, 1H), 2.00 (m, 1H), 2.39 (s, 3H), 2.52 (s, 3H), 2.59 (s, 3H), 2.68 (m, 1H), 4.06 (t, J=6.5 Hz, 2H), 4.93 (m, 2H), 4.99 (s, 1H), 5.69 (m, 1H), 6.17 (s, 1H NH), 7.23 (d, J=8 Hz, 2H), 7.58 (d, J=8 Hz, 2H); ¹³C (62.5 MHz, CDCl₃) δ21.3, 21.4, 25.5, 27.1, 31.1, 31.7, 34.2, 35.0, 36.7, 38.6, 41.6, 62.8, 113.8, 114.6, 127.3, 129.5, 136.7, 138.2, 143.8, 154.1, 163.0, 165.5, 167.8, 178.4. IR (film) cm⁻¹: 1726, 1640. HRMS Calcd for [M+1]⁺ C₂₉H₃₉N₃O₅S: calc 542.2688; found 542.2688

Compounds 45a and 45b

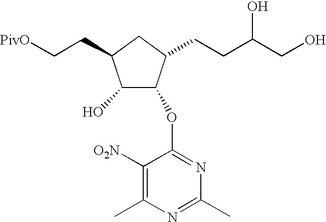

A solution of olefins 36a/36b (4.3 g, 9.81 mmol) in water-n-BuOH (40 mL each) was treated with MeSO₂NH₂ (0.933 g, 9.81 mmol) and AD-mix-β (14.7 g, 1.5 g/mmol substrate). The mixture was stirred rapidly for 1 h or until complete by TLC (KMnO₄), filtered, rinsed with n-BuOH (2×) and EtOAc (2×). The water was separated and the organics were concentrated. The mixture was redissolved in EtOAc, washed with brine, dried (MgSO₄) and concentrated in vacuo. The material was passed through a SiO₂ plug (EtOAc/Hex) to remove MeSO₂NH₂ contaminants. A yield of 3.4 g (75%) was obtained. ¹H (500 MHz, CDCl₃) (mixture of regioisomers) δ1.14 (s, 9H), 1.18 (s, 9H),1.3-2.2 (m, 20H), 2.48 (s, 6H), 2.58 (s, 3H), 2.60 (s, 3H), 3.35 (m, 2H), 3.58-3.66 (m, 4H), 3.86 (br m, 1H), 4.03-4.13 (br m, 4H), 4.28 (br m, 1H) 5.18 (br m, 1H), 5.69 (br q, J=4 Hz, 1H); ¹³C (125 MHz, CDCl₃) δ20.6, 26.1, 27.3, 31.6, 33.2, 33.5, 33.7, 37.1, 37.2, 38.8, 39.2, 40.3, 63.0, 63.3, 66.8, 66.9, 72.1, 72.2, 72.6, 72.8, 80.3, 82.8, 82.9, 85.2, 132.6, 160.2, 160.6, 160.7, 161.4, 168.4, 168.6, 178.7, 178.8. IR (film) cm⁻¹: 3384, 1724.

Compounds 46a and 46b

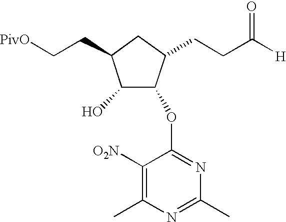

The mixture of regioisomers 45a/45b (3.5 g, 7.38 mmol) was dissolved in water/n-BuOH (40 mL each) and NaIO₄ (4.7 g, 22.1 mmol) was added. The reaction was judged complete in 30 min., by TLC. To the mixture was added 150 mL H₂O and 250 mL EtOAc and the layers were separated. The aqueous layer was back extracted with EtOAc (3×). The combined organics were dryed (MgSO₄) and concentrated. The crude aldehyde (2.99 g, 92.0%) was immediately taken on to the next step.

Compounds 47a and 47b

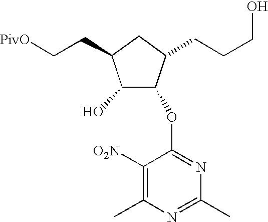

Aldehydes 46a/46b (2.0 g, 4.53 mmol) were dissolved in MeOH and cooled to 0° C. Solid NaBH₄ (0.20 g, 5.44 mmol) was added. After 15 min., a few drops of 10% HCl was added. The mixture was concentrated in vacuo, redissolved in EtOAc and washed with 10% HCl. Sodium hydroxide (6N) was added to the aqueous layer until pH 8 and was then extracted with EtOAc to remove additional product. The crude yield was 1.94 g, 96.6%. The mixture of regioisomers was taken on to the next step where they were separated by SiO₂ and,then fully characterized. HRMS Calcd for [M+1]⁺ C₂₁H₃₃N₃O₇: calc 440.2396; found 440.2397.

Compound 48b .

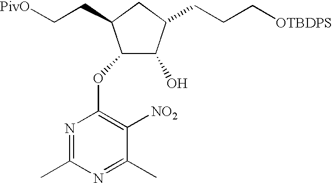

The mixture of diols 47a/47b (2.89 g, 6.52 mmol) was dissolved in CH₂Cl₂ (13 mL) and TEA (1.09 mL, 7.82 mmol), then DMAP (0.032 g, 0.26 mmol) was added. The solution was cooled to 0° C. A solution of TBDPSCl (1.79 mL, 6.84 mmol) in 13 mL CH₂Cl₂ was added dropwise to the alcohol. The reaction was complete after 3 h was washed with 10% HCl and H₂O, and dryed (MgSO₄). The regioisomers were separated by SiO₂ using 1:4 EtOAc/Hex to obtain 2.51 g, 77%. The regioisomers were isolated in a ˜1:1 mixture. ¹H (500 MHz, CDCl₃) δ1.05 (s, 9H), 1.17 (s, 9H), 1.47 (m, 1H), 1.50 (m, 1H), 1.60 (m, 2H), 1.67 (m, 2H), 1.81 (m, 1H), 1.87 (m, 1H), 1.97 (br m, 2H), 2.51 (s, 3H), 2.62 (s, 3H), 3.68 (m, 1H), 4.21 (br s, 1H), 4.97 (m, 2H), 5.18 (dd, J=3.5 Hz, J=8.5 Hz, 1H), 7.39 (m, 6H), 7.67 (m, 4H); ¹³C (62.5 MHz, CDCl₃) δ19.3, 20.5, 25.7, 26.1, 27.0, 27.2, 31.0, 33.0, 33.3, 37.1, 38.8, 39.9, 63.0, 64.1, 72.8, 85.5, 127.7, 129.6, 132.6, 134.2, 135.7, 160.2, 160.5, 168.4, 178.5. IR (film) cm⁻¹: 3440, 1726. HRMS Calcd for [M+1]⁺ C₃₇H₅₁N₃O₇Si: calc 678.3574; found 678.3576.

Compound 48a

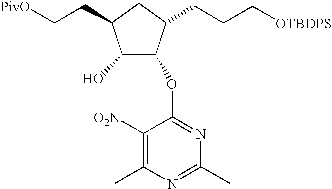

¹H (500 MHz, CDCl₃) δ1.01 (s, 9H), 1.21 (s, 9H), 1.32 (m, 1H), 1.54 (m, 4H), 1.65 (m, 1H), 1.72 (m, 1H), 1.95 (m, 1H), 2.0 (m, 1H), 2.18 (m, 1H), 2.50 (s, 3H), 2.58 (s, 3H), 3.60 (t, J=6.5 Hz, 2H), 3.83 (dt, J=4 Hz, J=9 Hz, 1H), 4.14 (m, 2H), 5.72 (t, J=4 Hz, 1H), 7.37 (m, 6H), 7.62 (m, 4H); ¹³C (62.5 MHz, CDCl₃) δ19.2, 20.5, 26.0, 26.4, 26.9, 27.3, 30.8, 33.4, 33.6, 38.8, 40.3, 53.5, 63.2, 63.7, 80.2, 82.8, 127.6, 129.6, 132.5, 134.0, 135.6, 160.5, 161.3, 168.2, 178.5. IR (film) cm⁻¹: 3477, 1725 . HRMS Calcd for [M+1]⁺ C₃₇H₅₁N₃O₇Si: calc 678.3574; found 678.3576.

Compound 50b

Alcohol 48b (1.05 g, 1.55 mmol) was dissolved in CH₂Cl₂ (0.4 mL) and mixed with TEMPO

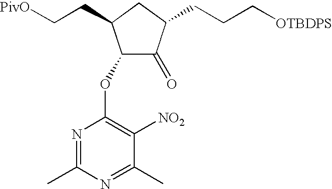

(2.4 mg, 0.015mmol) and aqueous KBr (1M, 2.58 mL) at −10° C. A solution of commercial bleach (5.25%) (2.58 mL, 1.70 mmol) containing NaHCO₃ (169 mg/10 mL) was added dropwise to the alcohol with rapid stirring. After completion (˜10 min), the reaction mixture was washed with 10% HCl containing NaI (150 mg/10 mL HCl), 10% Na₂S₂O₃, and H₂O. The organics were dryed (MgSO₄) and concentrated to afford the ketone in 86% yield (904 mg) ¹H (500 MHz, CDCl₃) δ1.05 (s, 9H), 1.18 (s, 9H), 1.47 (m, 1H), 1.63 (m, 2H) 1.85 (m, 3H), 2.03 (m, 2H), 2.44 (m, 2H) 2.51 (s, 3H), 2.56 (s, 3H), 3.67 (m, 1H), 4.15 (m, 2H), 5.52 (d, J=10 Hz, 1H) 7.40 (m, 6H), 7.66 (dd, J=1.5 Hz, J=8 Hz, 4H); ¹³C (62.5 MHz, CDCl₃) δ19.3, 20.5, 25.9, 26.8, 27.2, 28.0, 29.6, 30.2, 32.6, 35.8, 38.8, 43.1, 62.0, 63.4, 83.2, 127.6, 129.7, 132.0, 133.9, 135.6, 159.7, 160.6, 167.9, 178.5, 211.0. IR (Film) cm⁻¹: 1757, 1728. HRMS Calcd for [M+1]⁺ C₃₇H₄₉N₃O₇Si: calc 676.3418; found 676.3417.

Compound 50a

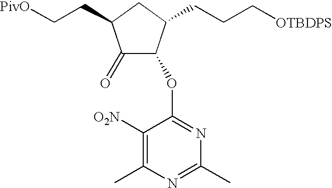

¹H (500 MHz, CDCl₃) δ1.02 (s, 9H), 1.19 (s, 9H), 1.54 (m, 2H), 1.67-1.80 (m, 4H), 2.12 (m, 1H), 2.22 (m, 1H), 2.35 (m, 1H), 2.51 (s, 3H), 2.59 (m, 1H), 2.59 (s, 3H), 3.65 (m, 2H), 4.15 (m, 2H), 5.87 (d, J=7.5 Hz, 1H), 7.39 (m, 6H), 7.63 (m, 4H); ¹³C (62.5 MHz, CDCl₃) δ19.2, 20.5, 23.5, 26.0, 26.9, 27.3, 28.7, 30.0, 30.2, 36.6, 38.3, 40.2, 62.1, 63.5, 80.2, 129.7, 129.7, 132.1, 133.8, 135.6, 159.6, 160.7, 168.1, 178.4, 211.6. IR (Film) cm⁻¹: 1757, 1726. HRMS Calcd for [M+1]⁺ C₃₇H₄₉N₃O₇Si: calc 676.3418; found 676.3417.

Compound 52

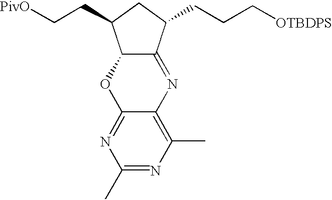

Nitro ketone 50a (or 50b) (100 mg, 0.147 mmol) was dissolved in EtOH (1 mL) and HOAc (84 μL, 1.47 mmol) and was heated to 70° C. Iron powder (−325 mesh, 41 mg, 0.735 mmol) was added. After 1 h, the hot reaction mixture was passed through Celite and evaporated under reduced pressure. The residue was dissolved in EtOAc and washed with sat. aqueous NaHCO₃. The organics were dryed (MgSO₄) and concentrated to yield 88.3 mg (90%) of the desired imine. The crude material was taken directly on to the next step. Note: this material degrades upon sitting over a period of days.

Compound 56

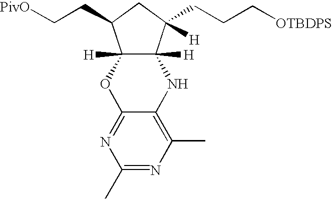

Imine 52 (88.3 mg, 0.140 mmol) was dissolved in MeOH (˜1 mL) and cooled to 0° C. NaBH₄ (6.4 mg, 0.168 mmol) was added as a solid in one portion. The reaction was complete in minutes and a drop of 10% HCl was added. The MeOH was removed in vacuo. The residue was dissolved in EtOAc and the solution was washed with sat. aq. NaHCO₃ to liberate 68.5 mg of the free amine. Preparative TLC resulted in isolation of material that was not any cleaner than the crude and mass recovery was poor. ¹H (500 MHz, CDCl₃) δ1.06 (s, 9H), 1.13 (s, 9H), 1.42 (m, 1H), 1.5-1.7 (m, 5H), 2.02, (m, 1H), 2.06 (m, 1H), 2.16 (m, 1H), 2.26 (s, 3H), 2.49 (s, 3H), 3.29 (br s, NH), 3.44 (br q, J=4 Hz, J=6 Hz, 1H), 3.69 (m, 2H), 4.10 (m, 2H), 4.38 (ddd, J=1 Hz, J=3.5 Hz, J=7 Hz, 1H), 7.39 (m, 6H), 7.65 (m, 4H); NOE(3.44): 4.38, 2.02; ¹³C (125 MHz, CDCl₃) δ18.4, 19.3, 24.6, 25.9, 27.0, 27.2, 30.9, 32.9, 33.0, 38.6, 39.0, 52.2, 63.1, 63.6, 84.4, 112.2, 127.7, 129.7, 133.9, 135.6, 149.0, 154.9, 156.3, 178.5. IR (film) cm⁻¹: 1801, 1725. HRMS Calcd for [M+1]⁺ C₃₇H₅₁N₃O₄Si: calc 630.3727; found 630.3727.

Compound 54

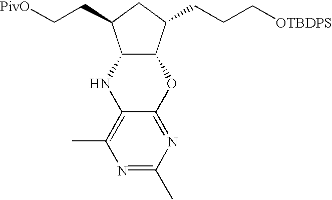

¹H (500 MHz, CDCl₃) δ1.04 (s, 9H), 1.13 (s, 9H), 1.62 (m, 6H), 1.8 (m, 3H), 2.08 (m, 1H), 2.29 (s, 3H), 2.51 (m, 3H), 3.40 (br m, 1H), 3:68 (m, 2H), 3.78 (br s, 1H), 4.20 (m, 2H), 4.22 (m, 1H), 3.37 (m, 6H), 7.65 (m, 4H); NOE(3.40): 4.20; ¹³C (125 MHz, CDCl₃) δ18.9, 19.3, 24.9, 25.1, 27.0, 30.9, 33.9, 34.4, 38.5, 38.6, 40.5, 60.6, 63.2, 63.9, 79.0, 119.9, 127.7, 129.6, 134.1, 135.6, 150.6, 155.8, 156.4, 178.6. IR (film) cm⁻¹: 1725. HRMS Calcd for [M+1]⁺ C₃₇H₅₁N₃O₄Si: calc 630.3727; found 630.3727.

Compound 59

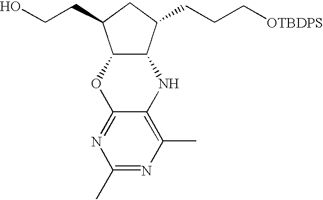

¹H (500 MHz, CDCl₃) δ1.05 (s, 9H), 1.38-1.93 (m, 8H), 2.00 (m, 1H), 2.23 (m, 1H), 2.25 (s, 1H), 2.47 (s, 3H), 3.30 (s, 1H), 3.32 (br s, 1H), 3.45 (br q, J=4 Hz, J=5.5 Hz, 1H), 3.70 (m, 4H), 4.43 (ddd, J=1 Hz, J=4 Hz,J=11.5 Hz, 1H), 7.37 (m, 6H), 7.64 (m, 4H); ¹³C (125 MHz, CDCl₃) δ18.7, 19.3, 24.9, 25.9, 31.0, 33.3, 37.6, 38.4, 39.0, 52.4, 61.3, 63.8, 84.8, 121.3, 127.8, 129.8, 133.9, 135.7, 149.6, 154.8, 156.4. IR (film) cm⁻¹: 3332, 3070, 1587, 1451.

Compound 57b

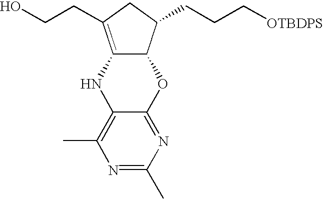

¹³C (125 MHz, CDCl₃) δ18.8, 19.2, 24.3, 26.9, 30.9, 32.7, 35.0, 41.6, 45.9, 63.8, 69.0, 81.3, 99.1, 99.3, 119.9, 127.5, 129.4, 134.0, 135.5, 150.4, 155.6, 156.8. IR (film) cm⁻¹: 3321, 1701, 1588, 1451.

It is evident from the above results and discussion that the subject invention provides an important new class of compounds that find use in a variety of different applications, including both therapeutic and diagnostic applications. Advantages provided by the subject compounds include increased half life and/or greater activity as compared to the peptidic counterparts. As such, the subject invention provides a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention.

What is claimed is:

1. A compound having a substantially rigid non-peptidic scaffold structure which mimics a binding site of Laminin Binding Protein.

2. The compound according to claim 1, wherein said molecule mimics the peptide Tyr-Ile-Gly-Ser-Arg binding site of Laminin Binding Protein.

3. The compound according to claim 1, further comprising arginine or an arginine-like group, tyrosine or a tyrosine-like group, isoleucine or an isoleucine-like group, and serine or a serine-like group, said substantially rigid non-peptidic scaffold positioning said groups in substantially the same manner as occurs in the peptide Tyr-Ile-Gly-Ser-Arg.

4. The compound according to claim 1, wherein said scaffold comprises a tricyclic heteroatom skeleton.

5. A peptidomimetic compound comprising a substantially rigid non-peptidic, scaffold, tyrosine or a tyrosine-like group, isoleucine or an isoleucine-like group, serine or a serine-like group, and arginine or an arginine-like group, said substantially rigid non-peptidic scaffold positioning said groups in substantially the same manner as occurs in the peptide Tyr-Ile-Gly-Ser-Arg.

6. The peptidomimetic compound according to claim 5, wherein said compound comprises the structure 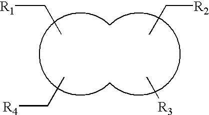
wherein R₁, R₂, R₃ and R₄ respectively comprise arginine or an arginine-like group, tyrosine or a tyrosine-like group, isoleucine or an isoleucine-like group, and serine or a serine-like group.

7. The peptidomimetic compound according to claim 6, wherein said compound further comprises the structure 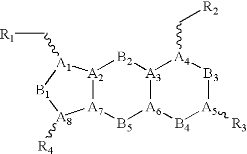
wherein A₁- A₈ comprise carbon or nitrogen, B₁- B₅ comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

8. The peptidomimetic compound according to claim 7, wherein said compound further comprises the structure 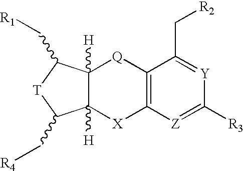
wherein Y and Z comprise carbon or nitrogen, T, Q and X comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

9. The peptidomimetic compound according to claim 6, wherein said compound comprises a structure selected from the group consisting of: 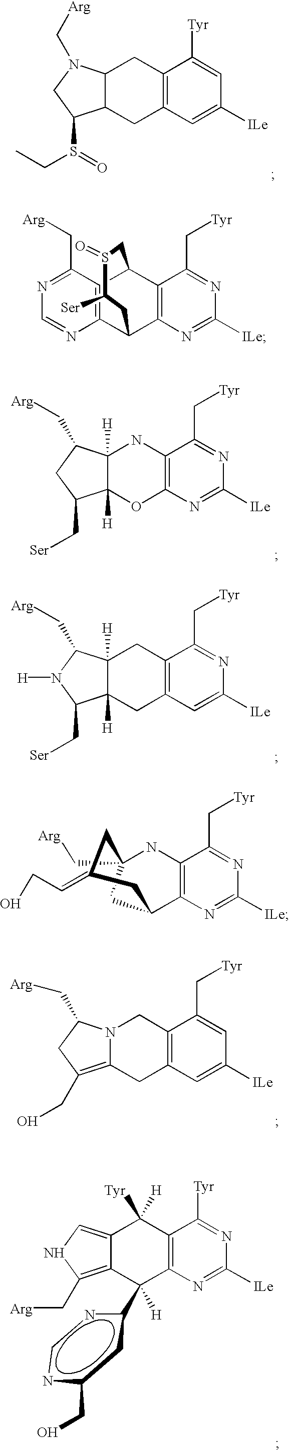 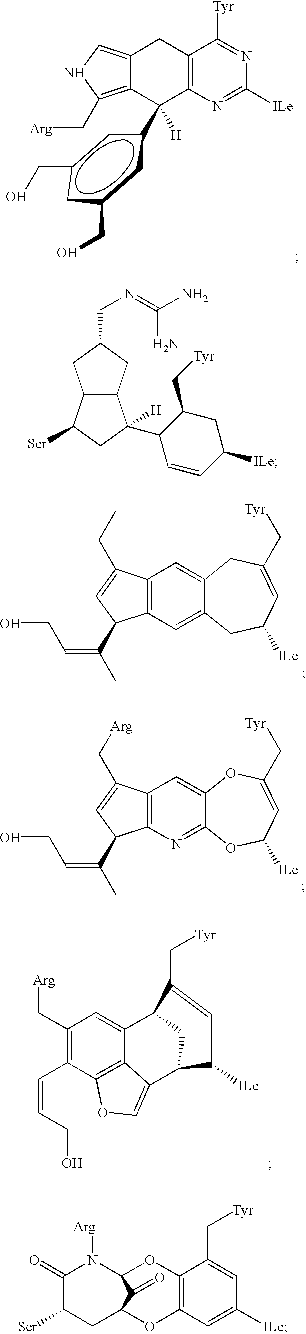 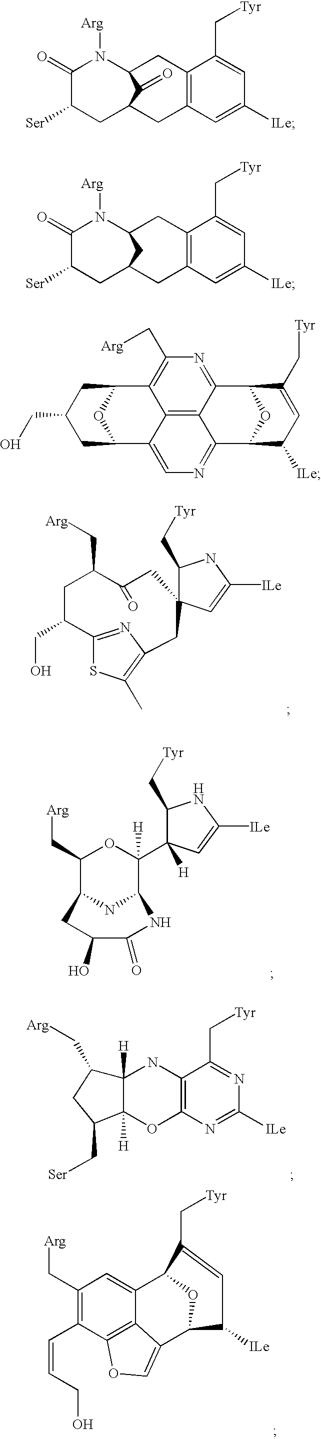 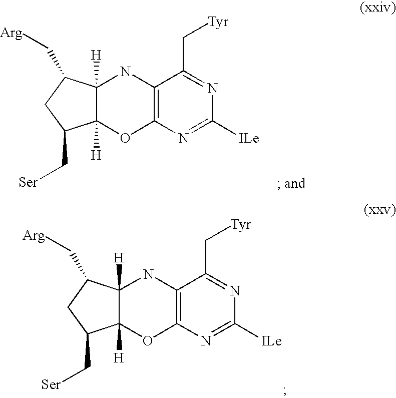
wherein Arg, Tyr, ILe and Ser respectively comprise arginine or an arginine-like group, tyrosine or a tyrosine-like group, isoleucine or an isoleucine-like group, and serine or a serine-like group.

10. A peptidomimetic compound comprising the structure 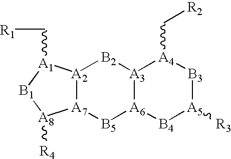
wherein A₁- A₈ comprise carbon or nitrogen, B₁- B₅comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

11. The peptidomimetic compound according to claim 10, wherein said compound further comprises the structure 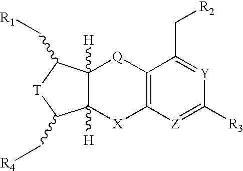
wherein Y and Z comprise carbon or nitrogen, T, Q and X comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

12. A pharmaceutical preparation of a compound having a substantially rigid non-peptidic scaffold structure which mimics a binding site of Laminin Binding Protein.

13. The pharmaceutical preparation according to claim 12, wherein said compound mimics the peptide Tyr-Ile-Gly-Ser-Arg binding site of Laminin Binding Protein.

14. The pharmaceutical preparation according to claim 12, wherein said compound further comprises a tyrosine-like group, an isoleucine-like group, a serine-like group, and an arginine-like group, said substantially rigid non-peptidic scaffold positioning said tyrosine-like group, said isoleucine-like group, said serine-like group and said arginine-like group in substantially the same manner as occurs in the peptide Tyr-Ile-Gly-Ser-Arg.

15. The pharmaceutical preparation according to claim 12, wherein said scaffold of said compound comprises a tricyclic heteroatom skeleton.

16. A pharmaceutical preparation comprising a peptidomimetic compound having the structure 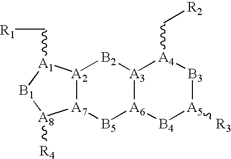
wherein A₁- A₈ comprise carbon or nitrogen, B₁-B₅ comprise carbon, nitrogen, oxygen or sulfur, and wherein R₁, R₂, R₃ and R₄ respectively comprise arginine or an arginine-like group, tyrosine or a tyrosine-like group, isoleucine or an isoleucine-like group, and serine or a serine-like group.

17. A method for binding the Tyr-Ile-Gly-Ser-Arg binding site of Laminin Binding Protein, comprising contacting said binding site with a peptidomimetic compound comprising a substantially rigid non-peptidic scaffold, a tyrosine-like group, an isoleucine-like group, a serine-like group, and an arginine-like group, said substantially rigid non-peptidic scaffold positioning said tyrosine-like group, said isoleucine-like group, said serine-like group and said arginine-like group in substantially the same manner as occurs in the peptide Tyr-Ile-Gly-Ser-Arg.

18. The method according to claim 17, wherein said binding site is present on a cell.

19. The method according to claim 18, wherein said cell is a disease cell.

20. The method according to claim 19, wherein said compound is coupled to an agent.

21. The method according to claim 20, wherein said agent is a therapeutic agent.

22. The method according to claim 20, wherein said agent is a label.

23. A method for treating a subject suffering from a cellular proliferative disease, said method comprising:
admininstering to said subject a therapeutically effective amount of a compound having a substantially rigid non-peptidic scaffold structure which mimics a binding site of Laminin Binding Protein.
